(19) United States
(12) Patent Application Publication
(10) Pub. No.: US 2022/0308075 A1
(43) Pub. Date: Sep. 29, 2022

(54) LIPID PROFILING METHODS FOR PREDICTING POSITIVE PREGNANCY OUTCOME

(71) Applicants: YISSUM RESEARCH DEVELOPMENT COMPANY OF THE HEBREW UNIVERSITY OF JERUSALEM LTD., Jerusalem (IL); HADASIT MEDICAL RESEARCH SERVICES & DEVELOPMENT LTD., Jerusalem (IL)

(72) Inventors: Arieh MOUSSAIEFF, Jerusalem (IL); Assaf BEN MEIR, Modiin (IL)

(21) Appl. No.: 17/608,412

(22) PCT Filed: May 7, 2020
(86) PCT No.: PCT/IL2020/050499

§ 371 Date: Nov. 2, 2021

Related U.S. Application Data
(60) Provisional application No. 62/844,898, filed on May 8, 2019.

(51) Int. Cl. G01N 33/92; G01N 33/82

(52) U.S. Cl. CPC G01N 33/92; G01N 33/82; G01N 2800/36

(57) ABSTRACT

The present invention relates to a method of determining the outcome of a pregnancy. More particularly, the invention relates to a method of profiling lipids in a fluid sample obtained from a female subject to assess parameters associated with a positive pregnancy outcome.

### FIELD OF THE INVENTION

The present invention relates to a method of determining the outcome of a pregnancy. More particularly, the invention relates to a method of profiling lipids in a fluid sample obtained from a female subject to assess parameters associated with a positive pregnancy outcome.

### BACKGROUND OF THE INVENTION

It has been estimated that human infertility, or the inability to conceive naturally, affects about 10% of couples of reproductive age around the world. Recent reports show that the main diagnoses for infertility include male infertility (20-30%), female infertility (20-35%), combined problems in both partners (25-40%), and unexplained infertility (10-20%).Unfortunately, the exact causes of infertility are numerous, and many of the specific mechanisms are still poorly understood. Nevertheless, over the last few decades, medical treatments, such as fertility medication, medical devices, surgery, and assisted reproductive technologies (ART) have made major improvements in correcting this condition.

The primary treatment option for many infertile couples often consists of in vitro fertilization (IVF), which is currently regarded as a successful technology with the number of IVF children currently well over 8 million worldwide. However, from its earliest history, IVF had low success rate in each treatment cycle, with an estimated 70% of IVF cycles fail to produce a live birth. Some of the reasons for a failed treatment include poor ovarian response (POR), age-related infertility, such as reduced ovarian reserve and decreased oocyte/embryo competence, recurrent implantation failure, or recurrent early pregnancy losses.

Therefore, to improve the success rate of embryo transfer, many of the treatment cycles performed involve the transfer of multiple embryos to increase the pregnancy rate but increases also the risk of multiple pregnancy, which is associated with increased maternal and fetal complications and significant costs of preterm birth. Numerous recent strategies to reduce the risks and increase the chances of a positive outcome in each IVF cycle (i.e., the birth of a single healthy baby), have been proposed in preparation for and during an IVF cycle. These include various tests for optimizing the outcome of IVF, such as identifying and selecting eggs or embryos with the greatest developmental potential. Interestingly, some tests, such as time lapse monitoring and the genetic assessment known as preimplantation genetic test—aneuploidy (PGT-A), have gained new information on early embryos potential. However, in spite of advancements in the field, at present, there is insufficient evidence to recommend the routine use of these new techniques.

The metabolome, which is the sum of all small molecules found in a biological sample, has been associated, in many independent studies, with the functional state of the cell or tissue examined, including that of the follicular fluid (FF), a liquid composed of blood plasma constituents that cross the blood follicular barrier, and secretions of follicle cells. The FF fills the follicular antrum and surrounds the oocyte, hence constructing its microenvironment. FF plays a key role in the nutritional and developmental support of the oocyte, promoting oocyte meiosis and development.

The lipidome, which is the set of lipids, is a segment of the metabolome. Despite increasing awareness of the importance of the lipid content of the oocyte microenvironment, a characterization of FF lipid composition and its relation to pregnancy outcome is still lacking.

There is a real need for a better understanding of the metabolic changes that occur in the stages of both egg and embryo maturation, as such knowledge could yield a promising IVF selection approach of noninvasively assessing the egg viability and developmental potential at a fraction of the costs and result in a successful treatment cycle. Recent advancement in freezing technologies resulted in considerably higher pregnancy rates. Thus, recent studies question the advantage of transferring fresh embryos, and suggest that in many cases, it would be advantageous to freeze the embryos and transfer them after the maternal uterus is recovered from the hormonal treatment and inflammatory response. Such an approach would encourage the development of cheaper stimulation protocols with less stress, discomfort and side effects. The analysis of follicular fluid may thus provide an important tool for informed decision making at the crossroads of transferring fresh embryos or freezing them. Furthermore, given that the follicular fluid composition is in many cases closely related to the blood plasma composition, biomarkers for pregnancy rate found in the follicular fluid may potentially be used for prediction of pregnancy outcome by using blood samples of IVF patients.

Given previous work that indicated conflicting roles played by specific lipid species in the development of the oocyte, it is a main object of the present invention to provide a method for characterizing the lipid composition in a fluid sample obtained from a subject and determining on the basis thereof the potential of oocyte and embryo development and the outcome of a pregnancy.

A second object of the present invention is to provide a method for determining a subject's source of infertility and assigning the subject to a particular treatment subgroup selected from male- or female-associated infertility, based on the lipid profile of said subject.

Another object of the present invention is to provide a method for selecting an oocyte suitable for use in IVF treatment from a plurality of candidate oocytes.

These and other objects of the invention will become apparent as the description proceeds.

### SUMMARY OF THE INVENTION

According to one aspect, the invention provides a method for determining the likelihood of a positive pregnancy outcome in a subject before or during fertilization treatment, comprising the steps of obtaining a fluid sample from a biological entity being the subject or an oocyte thereof, measuring in the said fluid sample the level of at least one lipid selected from the group of glycerolipids, phospholipids, lysophospholipids, sphingolipids, and cholesterol derivatives, comparing the measured level of said at least one lipid to the level of said at least one lipid in a predefined standard control, and determining the outcome of future or existing pregnancy based on said comparison.

According to one embodiment, the method further comprises the step of diagnosing the source of infertility for further treatment.

According to another embodiment, the method further comprises the step of providing the subject with a suitable treatment.

According to a further embodiment, the step of measuring further comprises measuring the levels of at least one of vitamin D, a derivative of vitamin D, several derivatives of vitamin D, or a combination of vitamin D and one or more of its derivatives.

According to a still further embodiment, the step of measuring further comprises measuring lipid levels from at least two different lipid groups, optionally at least three, optionally at least four, optionally of all five groups.

According to another embodiment, the step of comparing further comprises integrating the lipid levels from at least two, optionally at least three, optionally at least four, optionally from all five of the lipid groups, relative to predefined standard control levels, to provide a score reflecting the overall change across the lipid profile.

According to a further embodiment, the subject is a human or a mammal. According to a specific embodiment, the human subject has no history of infertility, unexplained infertility, known female infertility, known male factor infertility or is undergoing fertility treatment.

According to another embodiment, the biological entity is a fertilized oocyte originating from said subject or another subject, or is a genetically engineered oocyte.

According to another embodiment, the pregnancy outcome is considered positive if the female subject becomes pregnant.

According to another embodiment, the pregnancy outcome is considered positive if an oocyte develops into a viable fetus, the fetus continues to develop until its successful delivery, the pregnancy holds to full term, or the cycle of in vitro fertilization (IVF) is successful.

According to another embodiment, the fluid sample obtained from the biological entity is follicular fluid (FF) of a single oocyte, a pool of follicular fluids of several oocytes, or blood or plasma of a female subject that is either pregnant or is seeking to be pregnant in the future.

According to another embodiment, said suitable treatment is selected from selecting a fertilized oocyte for implantation, implanting a fertilized oocyte in a subject, starting or continuing with an IVF treatment cycle, treating said subject or their male partner for infertility, and using a surrogate mother for the pregnancy.

According to a further embodiment, a positive pregnancy outcome is indicated when the measured levels of at least one lipid selected from glycerolipids and cholesterol derivatives in said fluid sample are low relative to those of a predefined negative pregnancy outcome standard control. According to a specific embodiment, a positive pregnancy outcome is indicated when the fold change in the level of at least one lipid selected from glycerolipids and cholesteryl ester, measured in said fluid sample is 10% less relative to that of a predefined negative pregnancy outcome standard control. According to another specific embodiment, the glycerolipids are triacylglycerol (TAG), diacylglycerol (DAG), or a combination thereof.

According to a further embodiment, a positive pregnancy outcome is indicated when the measured levels of at least one lipid selected from phospholipids, lysophospholipids, sphingolipids, and vitamin D derivatives, are high in said fluid sample relative to that of a predefined negative pregnancy outcome standard control. According to a specific embodiment, a positive pregnancy outcome is indicated when the fold change in the level of at least one lipid selected from phospholipids, lysophospholipids, sphingolipids, or vitamin D derivatives, measured in said fluid sample is 10% more relative to that of a predefined negative pregnancy outcome standard control.

According to a further embodiment, a positive pregnancy outcome is indicated when the fold change in a level of a lipid selected from LysoPC(18:0), LysoPC(18:1), SM(d18:1/16:0), Lyso PC(18:1), LysoPC(18:2), PC(P-16:0/20:2) is at least 10% less than that of a predefined negative pregnancy outcome standard control.

According to a further embodiment, a positive pregnancy outcome is indicated when the fold change in a level of a lipid selected from TG(15:1/24:1/18:2), TG(14:1/16:0/20:0), TG(18:1/14:0/22:1), TG(14:1/20:0/21:0), TG(14:0/18:3/16:0), TG(18:0/24:0/20:4), TG(14:1/19:0/22:1), TG(18:0/16:0/18:0), TG(16:0/16:1/16:1), TG(20:0/20:3/22:0), TG(20:0/22:3/22:2), TG(16:1/18:0/20:0), TG(16:0/16:0/16:1), TG(14:0/16:0/16:1), TG(18:1/16:0/18:0), TG(16:1/18:1/18:1) is at least 10% greater than that of a predefined negative pregnancy outcome standard control.

According to another embodiment, the determination of the lipid level is done by using either a mass spectrometry (MS)-based technique or a non-MS-based technique.

According to another aspect, the invention provides a method for selecting an oocyte suitable for use in IVF treatment from a plurality of candidate oocytes, comprising the steps of obtaining follicular fluid of each candidate oocyte; measuring in said follicular fluid the level of at least one lipid selected from the group of glycerolipids, phospholipids, lysophospholipids, sphingolipids, and cholesterol derivatives; comparing the measured level of said at least one lipid to the level of said at least one lipid in a predefined standard control; identifying one or more oocytes likely to result in a positive pregnancy outcome based on said comparison; and selecting one or more of the oocytes identified for use in IVF treatment.

According to another aspect, the invention provides a method for determining whether to perform or continue an IVF procedure in a subject, comprising the steps of obtaining a blood sample from a subject; measuring in said blood sample the level of at least one lipid selected from the group of glycerolipids, phospholipids, lysophospholipids, sphingolipids, and cholesterol derivatives; comparing the measured level of said at least one lipid to the level of said at least one lipid in a predefined standard control; determining the outcome of future or existing pregnancy based on said comparison; and, if said subject is likely to have a positive pregnancy outcome, performing or continuing with the subject's IVF procedure.

### BRIEF DESCRIPTION OF THE DRAWINGS

FIG. 1A shows overlaid total ion chromatograms (TICs) of the metabolites detected by UPLC-MS, following sample preparation using different extraction systems.

FIG. 1B shows total number of all follicular fluid detected compounds. Performed in triplicate, and error bars represent standard error of the mean.

FIG. 1C shows number of highest abundance follicular fluid detected compounds. Performed in triplicate, and error bars represent standard error of the mean.

FIG. 2 shows a flow chart of participants in the study.

FIG. 3A shows a Partial least squares Discriminant Analysis (PLS-DA) of patients divided by the pregnancy outcome.

FIG. 3B shows a heat map of the top 100 lipids based on t-test.

FIG. 4A shows overlaid total ion chromatograms corresponding to FF samples prepared from older representative patients (over 39 years of age) with positive (blue) and negative (red) outcomes.

FIG. 4B shows overlaid total ion chromatograms corresponding to FF samples prepared from younger representative patients (under 32 years of age) with positive (blue) and negative (red) outcomes.

FIG. 4C shows the average abundance of all detected lipids in the FF of patients, where values from positive and negative outcome patients are plotted relative to one another. Error bars represent standard error of the mean. *, P<0.05; **, P<0.01; ***, P<0.001.

FIG. 4D shows the average abundance of all detected triacylglycerols (TAGs) in the FF of patients where values from positive and negative outcome patients are plotted relative to one another. Error bars represent standard error of the mean. *, P<0.05; **, P<0.01; ***, P≤0.001.

FIG. 4E shows the average abundance of all detected diacylglycerols (DAGs) in the FF of patients where values from positive and negative outcome patients are plotted relative to one another. Error bars represent standard error of the mean. *, P<0.05; **, P<0.01; ***, P≤0.001.

FIG. 4F shows the average abundance of all detected monoacylglycerols (MAGs) in the FF of patients where values from positive and negative outcome patients are plotted relative to one another. Error bars represent standard error of the mean. *, P<0.05; **, P<0.01; ***, P<0.001.

FIG. 4G shows the average abundance of all detected cholesteryl esters in the FF of patients where values from positive and negative outcome patients are plotted relative to one another. Error bars represent standard error of the mean. *, P<0.05; **, P<0.01; ***, P<0.001.

FIG. 4H shows the average abundance with respect to the following vitamin D derivatives in the FF of patients where values from positive and negative outcome patients are plotted relative to one another: 25-hydroxyvitamin D3; trihydroxyvitamin D3; 1alpha,25-dihydroxy-23-oxavitamin D3; 2beta-methoxy-1-alpha,25-dihydroxyvitamin D3; 1 alpha,25-dihydroxy-2beta-(5-hydroxypentoxy)vitamin D3/1 alpha,25-dihydroxy-2beta-(5-hydroxypentoxy) cholecalciferol; 24, 25-Dihydroxyvitamin D3; 1 alpha-hydroxy-26,27-dimethylvitamin D3/1alpha-hydroxy-26,27-dimethylcholecalciferol. Error bars represent standard error of the mean. *, P<0.05; **, P<0.01; ***, P<0.001.

FIG. 4I shows the average abundance of all detected lysophospholipids in the FF of patients where values from positive and negative outcome patients are plotted relative to one another. Error bars represent standard error of the mean. *, P<0.05; **, P<0.01; ***, P≤0.001.

FIG. 4J shows the average abundance of all detected phospholipids (PLs) in the FF of patients where values from positive and negative outcome patients are plotted relative to one another. Error bars represent standard error of the mean. *, P<0.05; **, P<0.01; ***, P<0.001.

FIG. 4K shows the average abundance of all detected sphingolipids (SLs) in the FF of patients where values from positive and negative outcome patients are plotted relative to one another. Error bars represent standard error of the mean. *, P<0.05; **, P<0.01; ***, P<0.001.

FIG. 4L shows the average abundance of all detected ceramides in the FF of patients where values from positive and negative outcome patients are plotted relative to one another. Error bars represent standard error of the mean. *, P<0.05; **, P<0.01; ***, P<0.001.

FIG. 4M shows the average abundance of all detected sphingomyelins in the FF of patients where values from positive and negative outcome patients are plotted relative to one another. Error bars represent standard error of the mean. *, P<0.05; **, P<0.01; ***, P<0.001.

FIG. 4N shows the average abundance of all detected glycosphingolipids in the FF of patients where values from positive and negative outcome patients are plotted relative to one another. Error bars represent standard error of the mean. *, P<0.05; **, P<0.01; ***, P<0.001.

FIG. 5A shows the average abundance of all detected triacylglycerols (TAGs) in the FF of younger patients (under 32 years of age) where values from positive and negative outcome patients are plotted relative to one another. Error bars represent standard error of the mean. *, P<0.05; **, P<0.01; ***, P<0.001.

FIG. 5B shows the average abundance of all detected diacylglycerols (DAGs) in the FF of younger patients (under 32 years of age) where values from positive and negative outcome patients are plotted relative to one another. Error bars represent standard error of the mean. *, P<0.05; **, P<0.01; ***, P<0.001.

FIG. 5C shows the average abundance of all detected sphingomyelins in the FF of younger patients (under 32 years of age) where values from positive and negative outcome patients are plotted relative to one another. Error bars represent standard error of the mean. *, P<0.05; **, P<0.01; ***, P<0.001.

FIG. 5D shows the average abundance of all detected phospholipids (PLs) in the FF of younger patients (under 32 years of age) where values from positive and negative outcome patients are plotted relative to one another. Error bars represent standard error of the mean. *, P<0.05; **, P<0.01; ***, P<0.001.

FIG. 5E shows the average abundance of all detected lysophospholipids in the FF of younger patients (under 32 years of age) where values from positive and negative outcome patients are plotted relative to one another. Error bars represent standard error of the mean. *, P<0.05; **, P<0.01; ***, P<0.001.

FIG. 5F shows the average abundance of all detected sphingolipids (SLs) in the FF of younger patients (under 32 years of age) where values from positive and negative outcome patients are plotted relative to one another. Error bars represent standard error of the mean. *, P<0.05; **, P<0.01; ***, P<0.001.

FIG. 5G shows the average abundance of all detected glycosphingolipids in the FF of younger patients (under 32 years of age) where values from positive and negative outcome patients are plotted relative to one another. Error bars represent standard error of the mean. *, P<0.05; **, P<0.01; ***, P<0.001.

FIG. 5H shows the average abundance of all detected ceramides in the FF of younger patients (under 32 years of age) where values from positive and negative outcome patients are plotted relative to one another. Error bars represent standard error of the mean. *, P<0.05; **, P<0.01; ***, P<0.001.

FIG. 5I shows the average abundance of all detected cholesteryl esters in the FF of younger patients (under 32 years of age) where values from positive and negative outcome patients are plotted relative to one another. Error bars represent standard error of the mean. *, P<0.05; **, P<0.01; ***, P<0.001.

FIG. 5J shows the average abundance of all detected vitamin D derivatives, as listed in the description of FIG. 4H, in the FF of younger patients (under 32 years of age) where values from positive and negative outcome patients are plotted relative to one another. Error bars represent standard error of the mean. *, P<0.05; **, P<0.01; ***, P<0.001.

FIG. 5K shows the average abundance of all detected triacylglycerols (TAGs) in the FF of older patients (over 39 years of age) where values from positive and negative outcome patients are plotted relative to one another. Error bars represent standard error of the mean. *, P<0.05; **, P<0.01; ***, P<0.001.

FIG. 5L shows the average abundance of all detected diacylglycerols (DAGs) in the FF of older patients (over 39 years of age) where values from positive and negative outcome patients are plotted relative to one another. Error bars represent standard error of the mean. *, P<0.05; **, P<0.01; ***, P<0.001.

FIG. 5M shows the average abundance of all detected sphingomyelins in the FF of older patients (over 39 years of age) where values from positive and negative outcome patients are plotted relative to one another. Error bars represent standard error of the mean. *, P<0.05; **, P<0.01; ***, P<0.001.

FIG. 5N shows the average abundance of all detected phospholipids (PLs) in the FF of older patients (over 39 years of age) where values from positive and negative outcome patients are plotted relative to one another. Error bars represent standard error of the mean. *, P<0.05; **, P<0.01; ***, P<0.001.

FIG. 5O shows the average abundance of all detected lysophospholipids in the FF of older patients (over 39 years of age) where values from positive and negative outcome patients are plotted relative to one another. Error bars represent standard error of the mean. *, P<0.05; **, P<0.01; ***, P<0.001.

FIG. 5P shows the average abundance of all detected sphingolipids (SLs) in the FF of older patients (over 39 years of age) where values from positive and negative outcome patients are plotted relative to one another. Error bars represent standard error of the mean. *, P<0.05; **, P<0.01; ***, P<0.001.

FIG. 5Q shows the average abundance of all detected glycosphingolipids in the FF of older patients (over 39 years of age) where values from positive and negative outcome patients are plotted relative to one another. Error bars represent standard error of the mean. *, P<0.05; **, P<0.01; ***, P<0.001.

FIG. 5R shows the average abundance of all detected ceramides in the FF of older patients (over 39 years of age) where values from positive and negative outcome patients are plotted relative to one another. Error bars represent standard error of the mean. *, P<0.05; **, P<0.01; ***, P<0.001.

FIG. 5S shows the average abundance of all detected cholesteryl esters in the FF of older patients (over 39 years of age) where values from positive and negative outcome patients are plotted relative to one another. Error bars represent standard error of the mean. *, P<0.05; **, P<0.01; ***, P<0.001.

FIG. 5T shows the average abundance of all detected vitamin D derivatives, as listed in the description of FIG. 4H, in the FF of older patients (over 39 years of age) where values from positive and negative outcome patients are plotted relative to one another. Error bars represent standard error of the mean. *, P<0.05; **, P<0.01; ***, P<0.001.

FIG. 6A shows a volcano plot representing lipids with a significant (FC>3 and FDR adjusted P value≤0.05) accumulation in the FF of positive and negative outcome patients. Lipids that are highly abundant in the FF of positive outcome patients are in blue; lipids with low levels in the FF of positive outcome patients are in red. Unidentified lipids are in pink. Arrows point to the 6 most discriminant lipids (graphs of their abundance are presented as extracted ion chromatograms in FIGS. 6B-6G). Discriminant lipids are numbered as follows: 1, PC(P-16:0/20:2); 3, LysoPC(18:2); 6, LysoPC(18:1); 7, SM(d18:1/16:0); 8, LysoPC(18:1); 9, LysoPC(18:0); 13, TG(16:1/18:0/20:0); 14, TG(18:0/16:0/18:0); 15, TG(16:0/16:0/16:1); 16, TG(15:1/24:1/18:2); 17, TG(18:1/14:0/22:1); 18, TG(14:1/19:0/22:1); 19, TG(16:0/16:1/16:1); 21, TG(18:1/16:0/18:0); 22, TG(14:1/16:0/20:0); 23, TG(14:1/20:0/21:0); 25, TG(18:0/24:0/20:4); 27, TG(14:0/16:0/16:1); 28, TG(14:0/18:3/16:0); 30, TG(20:0/20:3/22:0); 31, TG(20:0/22:3/22:2); 32, TG(16:1/18:1/18:1).

FIGS. 6B-6G show the chromatograms and relative abundance of 6 selected lipids with high potential as biomarkers of pregnancy outcome (data from all current cohort patients are included in the graphs).

FIG. 6B shows a representative extracted ion chromatogram of the 822.7539 m/z ion corresponding to TG(16:0/16:0/16:1) and a bar graph showing the average abundance of this lipid in the FF of patients where values from positive and negative outcome patients are plotted relative to one another. ***, P<0.001 by t-test.

FIG. 6C shows a representative extracted ion chromatogram of the 794.7221 m/z ion corresponding to TG(14:0/16:0/16:1) and a bar graph showing the average abundance of this lipid in the FF of patients where values from positive and negative outcome patients are plotted relative to one another. ***, P<0.001 by t-test.

FIG. 6D shows a representative extracted ion chromatogram of the 878.8158 m/z ion corresponding to TG(18:1/16:0/18:0) and a bar graph showing the average abundance of this lipid in the FF of patients where values from positive and negative outcome patients are plotted relative to one another. Abundances were quantified on the basis of the larger peak on the left side of the chromatogram. ***, P<0.001 by t-test.

FIG. 6E shows a representative extracted ion chromatogram of the unidentified 524.9069 m/z ion and a bar graph showing the average abundance of this putative lipid in the FF of patients where values from positive and negative outcome patients are plotted relative to one another. ***, P<0.001 by t-test.

FIG. 6F shows a representative extracted ion chromatogram of the 522.3559 m/z ion corresponding to lysoPC(18:1) and a bar graph showing the average abundance of this lipid in the FF of patients where values from positive and negative outcome patients are plotted relative to one another. Abundances were quantified on the basis of the larger peak on the right side of the chromatogram. ***, P<0.001 by t-test.

FIG. 6G shows a representative extracted ion chromatogram of the 1406.1425 m/z ion corresponding to SM(d18:1/16:0) and a bar graph showing the average abundance of this lipid in the FF of patients where values from positive and negative outcome patients are plotted relative to one another. ***, P<0.001 by t-test.

FIG. 6H shows a receiver operating characteristic (ROC) curve describing the predictive ability of 6 of the potential lipid biomarkers. The area under the curve was 0.85 with a standard error of 0.05. Blue circle represents cutoff point.

### DETAILED DESCRIPTION OF THE INVENTION

The inventors of the instant application have devised a novel and powerful analytical platform to delineate changes in the lipid network of the follicular fluid (FF) that are associated with pregnancy outcome. Namely, a lipidomics analysis of FF isolated from IVF patients was performed, and an association of a particular FF lipid composition with pregnancy rate was found. The lipid signature of FF that corresponded with a positive outcome of pregnancy included a lower accumulation of TAGS, diacylglycerols (DAGs) and cholesteryl esters, and a higher accumulation of PLs, SLs and vitamin D derivatives.

More specifically, by characterizing the association between the lipid composition of the FF and the outcome of a pregnancy, the present invention provides a unique analytical tool to both predict and assess the outcome of a pregnancy, performed by comparing the abundance of lipids from the FF of female subjects with positive and negative outcome.

As demonstrated in the examples further below, the inventors have surprisingly found a lipid remodeling of positive outcome FF, with a highly significant decrease (˜2 fold; P<0.001) in triacylglycerol levels and higher accumulation (10-50%; P<0.001) of membrane lipids groups—phospholipids and sphingolipids, as compared to negative outcome FF. This unique remodeling is utilized as a prognostic tool for initialing pregnancy and fertility treatments.

Further to the provision of these useful lipid biomarkers, the inventors have also identified additional major metabolic alterations in other lipid groups such as cholesteryl esters and derivatives of vitamin D, which showed lower and higher levels in the FF of positive outcome patients, respectively, as compared to negative outcome FF, thereby serving as additional biomarkers to predict and assess pregnancy outcome.

Overall, the body of data disclosed herein points to specific lipid species with a highly differential accumulation pattern in positive outcome FF relative to negative outcome FF, and therefore provides both unique lipid biomarkers and a profiling method for use in early pregnancy assessment, fertility treatments and indications of metabolic diseases.

In one aspect, the present invention discloses a method for determining the likelihood of a positive pregnancy outcome in a subject. The method comprises measuring in a fluid sample, obtained from the subject the level of at least one lipid species selected from the group of glycerolipids, phospholipids, lysophospholipids, sphingolipids, and cholesterol derivatives (which are also referred to herein as lipid biomarkers or prognostics).

One embodiment of the invention provides a method for determining the likelihood of a positive pregnancy outcome in a subject before or during fertilization treatment. The method comprises:

a. obtaining a fluid sample from a biological entity being the subject or an oocyte thereof;

b. measuring in the fluid sample obtained in step (a) the level of at least one lipid selected from the group of glycerolipids, phospholipids, lysophospholipids, sphingolipids, and cholesterol derivatives;

c. comparing the level of the at least one lipid measured in step (b) to the level of said at least one lipid in a predefined standard control; and

d. determining the outcome of future or existing pregnancy based on the comparison in (c).

According to a specific embodiment, the method further comprises the step of diagnosing the source of infertility for further treatment.

According to another specific embodiment, the method further comprises the step of providing the subject with a suitable treatment.

According to a specific embodiment, the fluid is follicular fluid (FF). Alternatively, the fluid is blood or cells in the FF.

According to another specific embodiment, the biological entity is a fertilized oocyte originating from said subject or another subject, or is genetically engineered oocyte.

According to a specific embodiment, a positive pregnancy outcome as considered in the instant discloser is any one of the following: the female subject becomes pregnant, an oocyte develops into a viable fetus, the fetus continues to develop until its successful delivery, the pregnancy holds to full term, or the cycle of in vitro fertilization (IVF) is successful.

According to a specific embodiment, suitable treatments that are provided on the basis of the lipid profiling methods of the present invention include but are not limited to selecting a fertilized oocyte for implantation, implanting a fertilized oocyte in a subject, starting or continuing with an IVF treatment cycle, treating said subject or their male partner for infertility, and using a surrogate mother for the pregnancy.

The expression “level relative to a predefined control” or “amount relative to a predefined control” or “abundance relative to a predefined control” are used herein interchangeably, and generally refer to a numerical representation of the concentration (amount) of a lipid in a biological sample (mg, μg, ng, pg, etc., per mL) relative to the concentration (amount) of that lipid in a control sample, such that the value is unitless. Furthermore, these terms can also refer to an absolute quantity in acceptable units as described in the art. The methods of the invention refer to the level or amount of the biomarker lipids in the sample relative to said predefined standard control. The term “measuring” or “determining” refers to a quantitative or qualitative determination of the amount or concentration of the biomarker lipid in a particular sample. For example, the determination of a level of a biomarker that is increasing or decreasing relative to a control can be based on a qualitative observation rather than on specific values of the biomarker in question.

A skilled practitioner will appreciate that the pregnancy assessment as disclosed herein could benefit many different types of female subjects presented with various backgrounds, such as but not limited to, individuals with no prior history of infertility, unexplained infertility, known female infertility, known male factor infertility, or other individuals who are otherwise undergoing fertility treatment.

Another specific embodiment of the invention is the provision of a test to determine whether a couple's infertility results from male-related factors or not. Specifically, in addition to performing standard hormonal tests, a physician may conduct the profiling methods disclosed herein on bodily samples of a female subject seeking to become pregnant and or to have a positive pregnancy outcome, in order to determine the cause of infertility, viz., to determine whether the infertility is caused by the female subject or not, and if not then direct the attention of the physician to further male infertility tests and treatments.

Triacylglycerols (also referred to as “triglycerides” or “triacylglycerides”; TAGs) are the most abundant lipids in oocytes, constituting over 50% of the lipidome, and provide a large potential energy reserve. TAG levels are inversely correlated to follicle size and positively correlated to the maternal body mass index (BMI) and levels of adipokines and pro-inflammatory cytokines in FF. The term “glycerolipids” as used herein refers preferably to triacylglycerol alone (TAG), diacylglycerols (DAG) alone, or a combination thereof. Specific examples are given below in Table 1.

Phospholipids (PLs) are the major component of biological membranes and are involved in the modulation of multiple cell functions, and cellular interactions. Scarce information is available on the PL content of the FF and its relation to embryo development. Several phosphatidylcholines (a type of PL) were found to show lower accumulation in poor ovarian responder patients. Choline and phosphocholine, which are precursors of phosphatidylcholines, also showed a differential accumulation in the FF of oocytes that developed into early cleavage-stage embryos. In seeming discrepancy to these findings, the total levels of PL was implied to be inversely correlated with higher percentages of fertilized oocytes. The term “phospholipids” (PL) as used herein refers to a glycerol backbone linked to two fatty acid (also known as fatty acyl) chains and a phosphate group, where either one or both of the fatty acids or phosphate groups is modified. Specific examples of PL are given below in Table 1.

The term “lysophospholipids” refers to any derivative of a phospholipid in which one of the fatty acyl chains has been removed by hydrolysis. Specific examples of lysophospholipids are given below in Table 1.

Sphingolipids (SLs) form another notable group of bioactive lipids, some of them known to be mediating or regulating proliferative responses, growth inhibition, apoptosis, differentiation and senescence, and cell motility. The FF levels of four SL species were previously positively correlated with oocyte cleavage rate. The term “sphingolipids” as used herein refers to of lipids containing a backbone of sphingoid bases, a set of aliphatic amino alcohols that includes sphingosine. Specific examples are given below in Table 1.

Cholesterol derivatives have also been implicated in female fertility; however, previous work on this group of lipids was mostly focused on gonadal hormones and lipoproteins. Cholesterol derivatives in accordance with the present invention are cholesteryl esters.

Furthermore, recent studies have also suggested a relationship between the abundance of molecular species of the vitamin D subgroup of cholesterol derivatives and in vitro fertilization (IVF) outcomes. However, current literature offers conflicting evidence for the levels and the roles of these lipids in this context. Particularly, in a few recent examples, vitamin D abundance was positively correlated to success of an IVF cycle, while others found that FF level of 25-hydroxyvitamin D correlates negatively with the oocyte's ability to undergo fertilization and subsequent preimplantation embryo development, or that lower follicular 25-hydroxyvitamin D concentrations predicted a better response to ovarian stimulation.

TABLE 1
Lipid groups and examples of representative species.

| | Representative | |
|---|---|---|
| | Species putatively | Empirical |
| Group | identified | Formula |
| Lyso- | Lyso PC(18:1) | C₂₆H₅₂NO₇P |
| phospholipids | LysoPC(18:0) | C₂₆H₅₄NO₇P |
| | LysoPC(18:1) | C₂₆H₅₂NO₇P |
| | LysoPC(18:2) | C₂₆H₅₀NO₇P |
| Phospholipids | PC(P-16:0/20:2) | C₄₄H₈₄NO₇P |
| Sphingomyelin | SM(d18:1/16:0) | C₃₉H₇₉N₂O₆P |
| (sphingolipid) | | |
| Glycerolipids | TG(14:0/16:0/16:1) | C₄₉H₉₂O₆ |
| | TG(14:0/18:3/16:0) | C₅₁H₉₂O₆ |
| | TG(14:1/16:0/20:0) | C₅₃H₁₀₀O₆ |
| | TG(14:1/19:0/22:1) | C₅₈H₁₀₈O₆ |
| | TG(14:1/20:0/21:0) | C₅₈H₁₁₀O₆ |
| | TG(15:1/24:1/18:2) | C₆₀H₁₀₈O₆ |
| | TG(16:0/16:0/16:1) | C₅₁H₉₆O₆ |
| | TG(16:0/16:1/16:1) | C₅₁H₉₄O₆ |
| | TG(16:1/18:0/20:0) | C₅₇H₁₀₈O₆ |
| | TG(16:1/18:1/18:1) | C₅₃H₁₀₂O₆ |
| | TG(18:0/16:0/18:0) | C₅₅H₁₀₆O₆ |
| | TG(18:0/24:0/20:4) | C₆₅H₁₁₈O₆ |
| | TG(18:1/14:0/22:1) | C₅₇H₁₀₆O₆ |
| | TG(18:1/16:0/18:0) | C₅₅H₁₀₄O₆ |
| | TG(20:0/20:3/22:0) | C₆₅H₁₂₀O₆ |
| | TG(20:0/22:3/22:2) | C₆₇H₁₂₀O₆ |

In a specific embodiment, the lipid levels to be measured comprise also the level of vitamin D, a derivative of vitamin D or several derivatives of vitamin D or a combination of vitamin D, and one or more of its derivatives. Specific examples of vitamin D derivatives which may be measured include, but are not limited to, 25-hydroxyvitamin D3, trihydroxyvitamin D3, 1alpha,25-dihydroxy-23-oxavitamin D3, 2beta-methoxy-1-alpha,25-dihydroxyvitamin D3, 1alpha,25-dihydroxy-2beta-(5-hydroxypentoxy)vitamin D3/1alpha,25-dihydroxy-2beta-(5-hydroxypentoxy)cholecalciferol, 24,25-Dihydroxyvitamin D3, and 1alpha-hydroxy-26,27-dimethylvitamin D3/1alpha-hydroxy-26,27-dimethylcholecalciferol.

The measurement of a particular lipid species' level in a fluid from a subject can be done by a number of approaches that are well-known in the art, and is preferably done according to the present invention by determining the levels of said lipid species relative to a predefined standard control level. This may be achieved by using at least one of several known quantitative techniques, such as, but not limited to either non-mass spectrometry (MS) based techniques (e.g., antibody-based, chromatography-based, nuclear magnetic resonance-based, Raman spectroscopy-based, etc.) or MS-based ones, as demonstrated in the examples herein further below. As should be apparent to the skilled practitioner in the field, said predefined standard control level may be easily obtained from prior knowledge or may be empirically derived by measuring, with quantitative methods as discussed above, the average levels of lipids in individuals with a known state of fertility.

According to some embodiments of the invention, the assessment of the likelihood of a positive pregnancy outcome is based on the determination of the lipid levels of at least two different lipid groups, optionally at least three, optionally at least four, optionally of all five groups mentioned above.

According to other particular embodiments of the instant invention, a determination that indicates a positive pregnancy outcome is one where the measured lipid levels of glycerolipids (TAG, DAG or their combination) and/or cholesteryl esters are low, while those of phospholipids, lysophospholipids, sphingolipids and/or vitamin D derivatives are high, relative to a predefined negative pregnancy outcome standard control.

In some embodiments, a determination that indicates a positive pregnancy outcome is one where the measured lipid levels of glycerolipids (TAG, DAG or their combination) and/or cholesteryl esters are 10% less relative to a predefined negative pregnancy outcome standard control, while the lipid levels of phospholipids, lysophospholipids, sphingolipids and/or vitamin D derivatives are 10% more relative to a predefined negative pregnancy outcome standard control.

In more specific embodiments, a positive pregnancy outcome is indicated when the fold change in a level of either one of the lipids LysoPC(18:0), LysoPC(18:1), SM(d18:1/16:0), Lyso PC(18:1), LysoPC(18:2), PC(P-16:0/20:2) or their diagnostic ions is at least 10% less than that of a predefined negative pregnancy outcome standard control, or, alternatively, when the fold change in the level of either one of the lipids TG(15:1/24:1/18:2), TG(14:1/16:0/20:0), TG(18:1/14:0/22:1), TG(14:1/20:0/21:0), TG(14:0/18:3/16:0), TG(18:0/24:0/20:4), TG(14:1/19:0/22:1), TG(18:0/16:0/18:0), TG(16:0/16:1/16:1), TG(20:0/20:3/22:0), TG(20:0/22:3/22:2), TG(16:1/18:0/20:0), TG(16:0/16:0/16:1), TG(14:0/16:0/16:1), TG(18:1/16:0/18:0), TG(16:1/18:1/18:1) or their diagnostic ions is at least 10% greater than that of a predefined negative pregnancy outcome standard control.

Conversely, a result which may also indicate a positive pregnancy outcome in a subject is one where the measured levels of said lipids match those of a predefined positive outcome standard control.

In preferred embodiments of the present invention, a positive pregnancy outcome is indicated for a subject exhibiting, relative to a predefined negative pregnancy outcome standard control, a lipid level in accordance with that of any one of the particular lipids demonstrated, as in the examples further herein and summarized in Table 2 below, to have a highly differential accumulation level relative to a predefined negative pregnancy outcome standard control, as signified by said lipid's respective fold change value, preferably given as a logarithm to the base 2 (log₂(FC)).

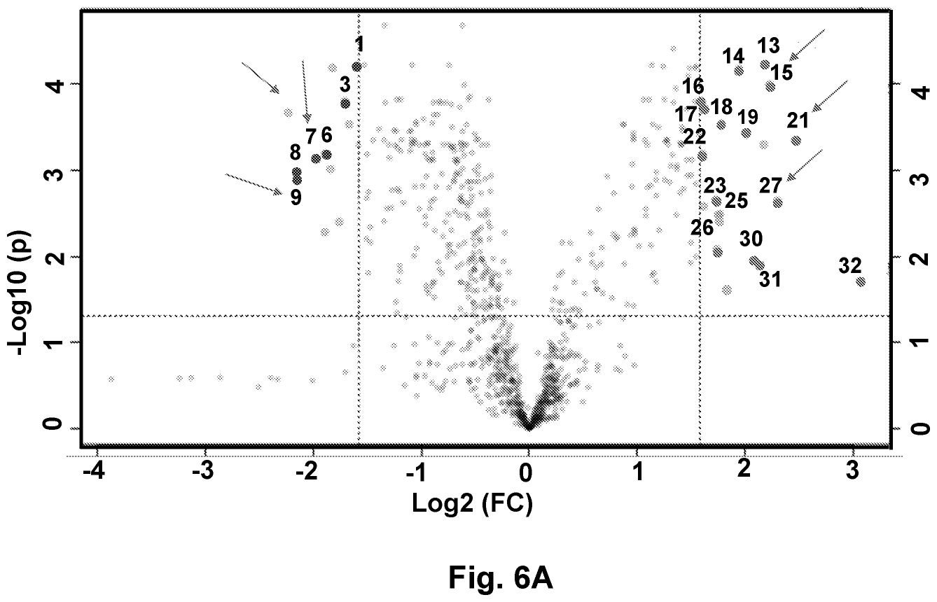

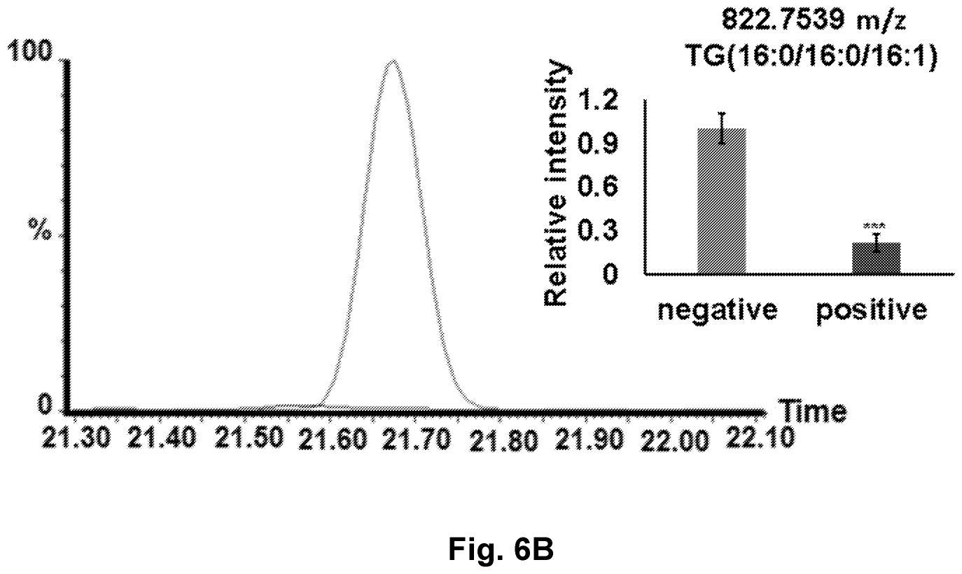

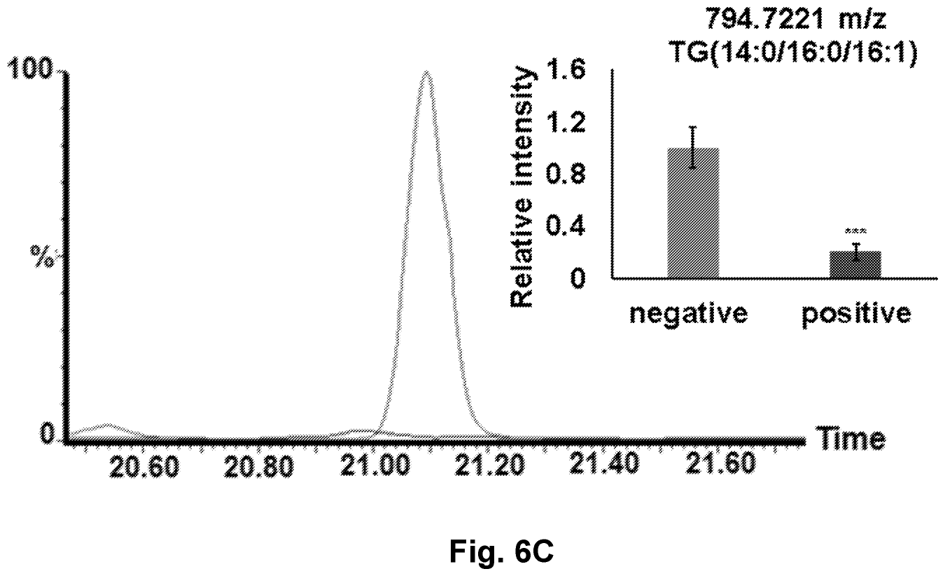

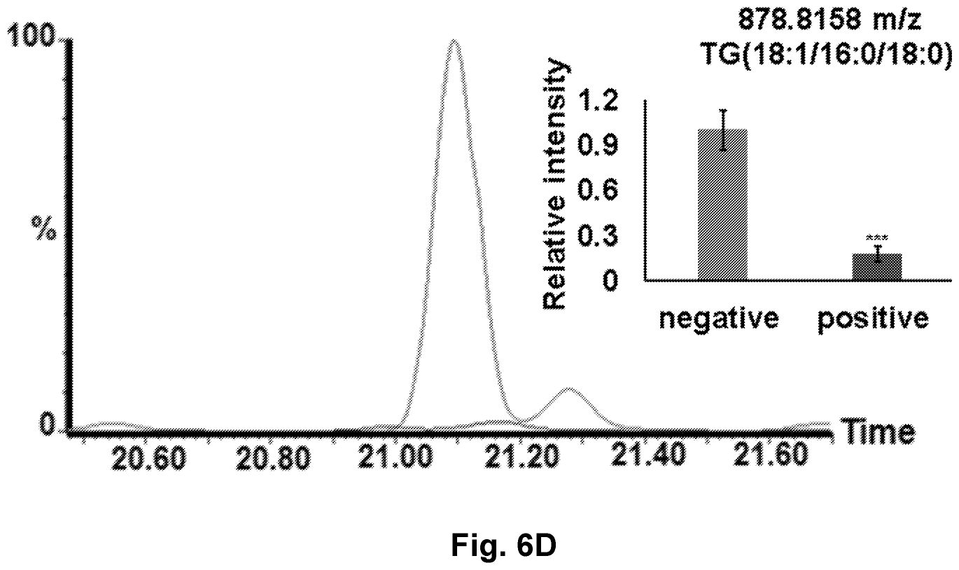

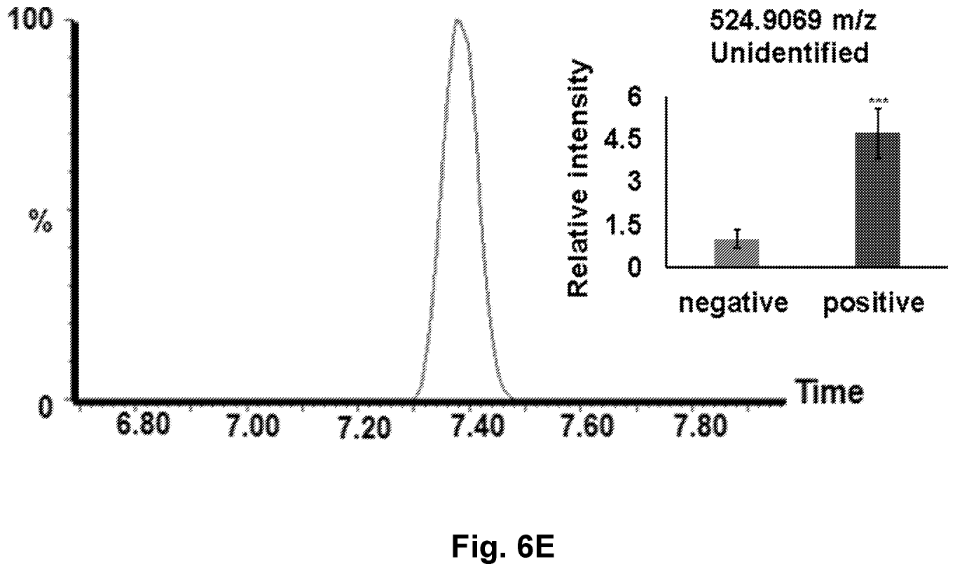

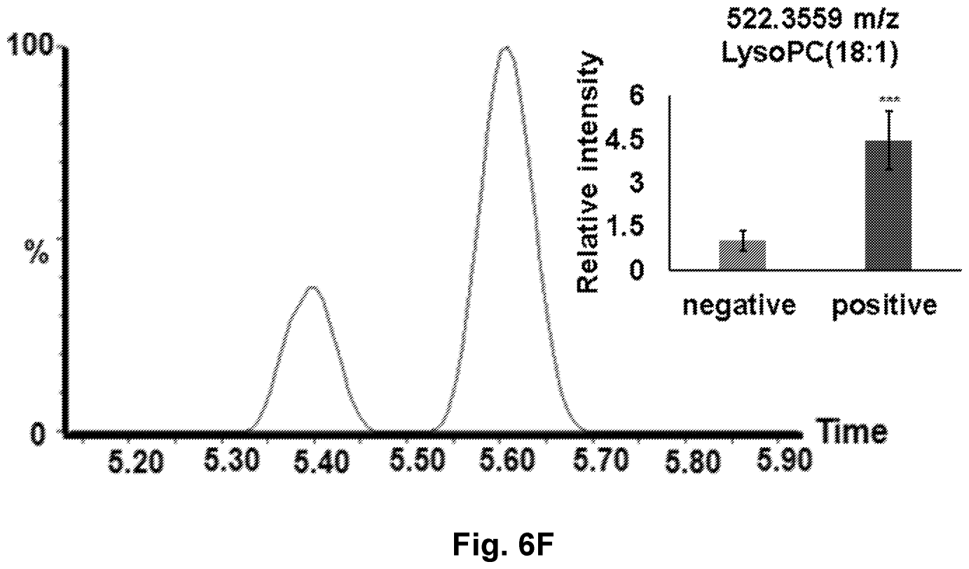

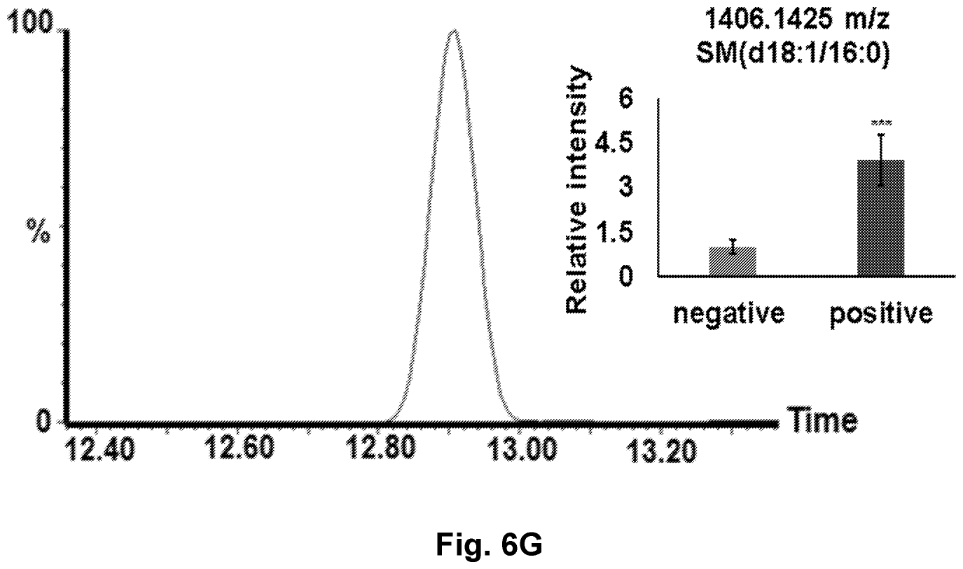

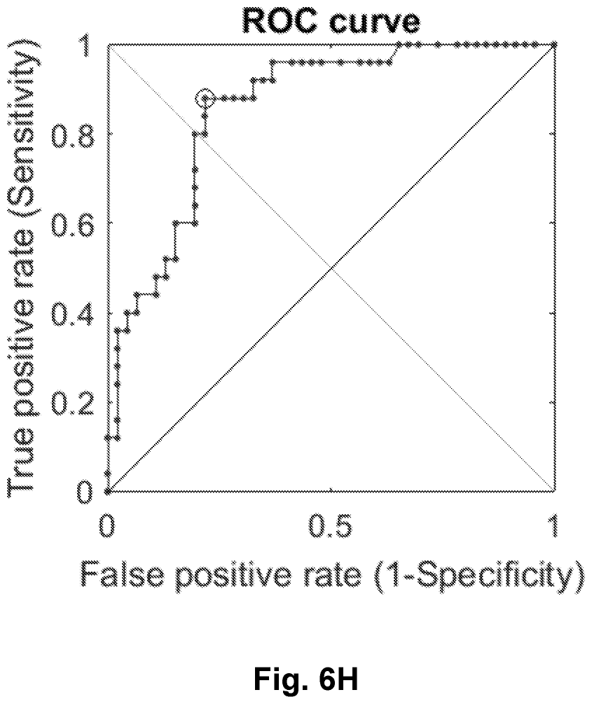

TABLE 2
Putatively identified, differentially accumulated lipids in the FF of positive
outcome patients as depicted in FIG. 6

| | | Retention | | | Mass | | |
|---|---|---|---|---|---|---|---|
| Putative | | time | Empirical | Diagnostic | Error | | |
| Identification | m/z | (mm) | Formula | ions | (ppm) | log₂(FC) | q |
| Unidentified | 524.91 | 7.47 | | | | −2.23 | 0.0002 |
| LysoPC(18:0) | 524.37 | 6.36 | C₂₆HNO₇P | 524.4 | 2.09 | −2.16 | 0.0013 |
| | | | | 506.3; | | | |
| | | | | 492.6; | | | |
| | | | | 164.07; | | | |
| | | | | 104 | | | |
| LysoPC(18:1) | 522.36 | 5.64 | C₂₆HNO₇P | 522.3; | 0.19 | −2.16 | 0.0011 |
| | | | | 504.3; | | | |
| | | | | 184.07; | | | |
| | | | | 104 | | | |
| SM(d18:1/16:0) | 1406.14 | 13.05 | C₃₉H₇₉N₂O₆P | 1406.14; | 0.06 | −1.97 | 0.0007 |
| | | | | 164.07; | | | |
| | | | | 665.56; | | | |
| | | | | 264 | | | |
| Unidentified | 763.47 | 13.45 | C₂₀HO₆ | | 3.07 | −1.69 | 0.0053 |
| Lyso PC(18:1) | 1043.70 | 6.36 | C₂₆HNO₇P | 1043.9; | −0.47 | −1.69 | 0.0006 |
| | | | | 164.07; | | | |
| | | | | 104 | | | |
| Unidentified | 783.91 | 13.48 | | | | −1.84 | 0.0010 |
| Unidentified | 1044.71 | 6.14 | | | | −1.82 | 0.0001 |
| Unidentified | 759.46 | 13.67 | C₄₃HO₈P | | | −1.76 | 0.0040 |
| LysoPC(18:2) | 520.61 | 5.44 | C₂₆H₅₀NO₇P | 520.6; | 4.60 | −1.71 | 0.0002 |
| | | | | 502.3; | | | |
| | | | | 184.07; | | | |
| | | | | 104 | | | |
| Unidentified | 1005.69 | 7.45 | | | | −1.67 | 0.0003 |
| PC(P-16:0/20:2) | 1562.18 | 14.30 | C₄₄H₈₄NO₇P | 1562.18; | −0.90 | −1.60 | 0.0001 |
| | | | | 184.07; | | | |
| | | | | 464.3; | | | |
| | | | | 308 | | | |
| TG(15:1/24:1/18:2) | 1008.89 | 21.62 | C₆₀H₁₀₈O₆ | 924.81; | 2.35 | 1.60 | 0.0002 |
| | | | | 625.5; | | | |
| | | | | 227 | | | |
| TG(14:1/16:0/20:0) | 850.79 | 22.22 | C₅₃H₁₀₀O₆ | 850.8; | −0.95 | 1.61 | 0.0006 |
| | | | | 577.5; | | | |
| | | | | 239.2 | | | |
| TG(18:1/14:0/22:1) | 904.83 | 21.70 | C₅₇H₁₀₀O₆ | 904.82; | −3.32 | 1.61 | 0.0002 |
| | | | | 265.25; | | | |
| | | | | 605.55 | | | |
| Unidentified | 959.13 | 21.77 | | | | 1.62 | 0.0026 |
| TG(14:1/20:0/21:0) | 920.86 | 21.85 | C₅₈H₁₁₀O₆ | 920.9; | −1.43 | 1.73 | 0.0022 |
| | | | | 623.5 | | | |
| TG(14:0/18:3/16:0) | 818.72 | 20.57 | C₅₁H₉₂O₆ | 818.7; | −1.26 | 1.74 | 0.0083 |
| | | | | 573.48; | | | |
| | | | | 522.4; | | | |
| | | | | 263.2 | | | |
| Unidentified | 1058.91 | 21.40 | C₆₅H₁₁₈O₆ | | | 1.75 | 0.0033 |
| TG(18:0/24:0/20:4) | 1058.91 | 21.18 | C₆₅H₁₁₈O₆ | 1058.9; | 0.40 | 1.77 | 0.0033 |
| | | | | 711.6; | | | |
| | | | | 351.36 | | | |
| TG(14:1/19:0/22:1) | 918.85 | 21.47 | C₅₈H₁₀₈O₆ | 918.8; | −1.79 | 1.78 | 0.0003 |
| | | | | 603.5; | | | |
| | | | | 675.6 | | | |
| Unidentified | 1036.93 | 22.01 | C₆₃H₁₂₀O₆ | | | 1.83 | 0.0250 |
| TG(18:0/16:0/18:0) | 904.83 | 21.14 | C₅₅H₁₀₀O₆ | 904.83; | −1.02 | 1.95 | 0.0001 |
| | | | | 265.25; | | | |
| | | | | 604.54 | | | |
| TG(16:0/16:1/16:1) | 820.74 | 21.17 | C₅₁H₉₆O₆ | 820.73; | −1.15 | 2.01 | 0.0004 |
| | | | | 547.47; | | | |
| | | | | 549.48; | | | |
| | | | | 239.23; | | | |
| | | | | 237.22 | | | |
| TG(20:0/20:3/22:0) | 1060.93 | 21.67 | C₆₅H₁₂₀O₆ | 1060.9; | 1.53 | 2.08 | 0.0111 |
| | | | | 685.61; | | | |
| | | | | 657.5; | | | |
| | | | | 397.36 | | | |
| TG(20:0/22:3/22:2) | 1084.93 | 21.33 | C₆₇H₁₂₀O₆ | 1084.9; | 1.66 | 2.10 | 0.0116 |
| | | | | 687.62; | | | |
| | | | | 685.61 | | | |
| Unidentified | 1056.89 | 20.88 | C₆₉H₁₁₄O₆ | | | 2.18 | 0.0005 |
| TG(16:1/18:0/20:0) | 906.85 | 21.69 | C₅₇H₁₀₈O₆ | 906.84; | −0.89 | 2.18 | 0.0001 |
| | | | | 323.3; | | | |
| | | | | 577.51 | | | |
| TG(16:0/16:0/16:1) | 822.75 | 21.68 | C₅₁H₉₆O₆ | 822.75; | −0.82 | 2.23 | 0.0001 |
| | | | | 549.48 | | | |
| TG(14:0/16:0/16:1) | 794.72 | 21.10 | C₄₉H₉₂O₆ | 794.7; | −1.46 | 2.30 | 0.0023 |
| | | | | 521.45; | | | |
| | | | | 523.47; | | | |
| | | | | 549.5; | | | |
| | | | | 313.27 | | | |
| TG(18:1/16:0/18:0) | 878.82 | 21.10 | C₅₅H₁₀₄O₆ | 878.82; | −1.50 | 2.46 | 0.0004 |
| | | | | 265.2; | | | |
| | | | | 239.2; | | | |
| | | | | 578.5 | | | |
| TG(16:1/18:1/18:1) | 876.80 | 20.54 | C₅₃H₁₀₂O₆ | 876.80; | −1.69 | 3.07 | 0.0194 |
| | | | | 265; | | | |
| | | | | 235.05 | | | |

In some embodiments, the determined lipid levels relative to the predefined pregnancy outcome control standard may be given in the form of a general score formed by the integration of the relevant lipid levels to reflect the overall change across the lipid profile. The integration can also be performed in a weighted manner, in which the levels of lipids from at least two, optionally at least three, optionally at least four, and preferably all five of the lipid groups listed above, relative to said predefined control levels are weighed into a final score, taking into consideration how low the level of glycerolipids and cholesteryl esters is and how high the level of phospholipids, lysophospholipids, and sphingolipids is compared to a said standard control. Alternatively, the result of the assessment can be given in a binary yes or no manner, which reflects said general score.

A skilled practitioner should appreciate that the methods disclosed herein are suitable both for human purposes as well as for veterinary use, as in IVF of cattle or a pet animal, and, hence, the “subject” may be a human or a mammal (such as but not limited to a mammal used in farming, or a rare species that is bred in zoos).

In another embodiment of the present invention, the likelihood of a positive pregnancy outcome is tested with parameters selected from a group consisting of: the chance that an oocyte will develop into a viable fetus, the chance that the fetus will continue to develop until its successful delivery, the chance that a female subject (human or mammal) will become pregnant, the chance that the pregnancy will hold to full term, and the chance that a cycle of IVF will be successful.

A skilled person will surely be cognizant of the fact that in addition to follicular fluid, many other types of fluids obtained from a biological entity, e.g., bodily fluids, may be analyzed via the methods of the present invention to yield relative lipid levels indicative of a particular pregnancy outcome, as demonstrated for the analysis of follicular fluid in the examples further herein below. Other examples of useful fluids obtained from a biological entity suited for such profiling methods as disclosed in the instant invention include, but are not limited to, follicular fluid of a single oocyte, a pool of follicular fluids of several oocytes, or blood and or plasma of a female subject that is either pregnant or seeking to be pregnant in the future.

Where the fluid to be analyzed is follicular fluid surrounding a single oocyte, the assessment method disclosed herein can help select the oocyte with the highest potential and best chances of leading to a positive pregnancy outcome. Such an oocyte is given the highest score and is returned to the uterus of a female subject in the framework of an IVF treatment.

Accordingly, another aspect of the present invention concerns a method for selecting an oocyte suitable for use in IVF treatment from a plurality of candidate oocytes, in which the follicular fluid (FF) obtained from each candidate oocyte is subjected to the lipid profiling method disclosed herein (i.e., measuring in the follicular fluid obtained the level of at least one lipid selected from the group of glycerolipids, phospholipids, lysophospholipids, sphingolipids, and cholesterol derivatives, comparing said measured lipid level to that in a predefined standard control, and identifying one or more oocytes likely to result in a positive pregnancy outcome based on said comparison), and one or more of the oocytes which fluid has the highest scores (as measured by the method specified above) is selected and used in IVF treatment.

In another aspect of the invention where the fluid to be analyzed is a fluid pooled from several oocytes extracted for IVF purposes in either a human or another mammal subject, the method disclosed herein may advise the skilled practitioner of the likelihood of obtaining a positive outcome in a future pregnancy, as well as of whether the chance of a positive outcome is high enough to proceed with an ongoing IVF procedure, taking into consideration costs and medical risks as well.

In another aspect of the invention where the fluid to be analyzed is a fluid pooled from specific oocytes extracted for IVF purposes in either a human or another mammal subject, the method disclosed herein may advise the skilled practitioner of the developmental potential of each oocyte, and a means for choosing the one with the highest developmental potential.

The methods provided herein may help physicians in the following therapeutic steps to be considered:
1. Testing for male or female infertility and treating accordingly.
2. Testing whether a couples' infertility stems from male or female factors and treating accordingly.
3. Evaluating the chances of a successful pregnancy.
4. Using fresh embryos or freezing them.
5. Recommending surrogate mother in extreme cases.
6. Determining the best timing for a successful IVF treatment (important given the costs per cycle).

Where the fluid is blood or plasma of a female subject (human or mammal) already pregnant, about to become pregnant, or about to undergo IVF treatment, the method can help, alone or together with other methods and tests, to evaluate the chances of the pregnancy reaching successful delivery, the chances of the female subject of becoming pregnant or succession in IVF treatment.

Thus, in a further aspect, the present invention concerns a method for determining whether to perform or continue an IVF procedure in a subject, in which a blood sample obtained from said subject is subjected to the lipid profiling method disclosed herein (i.e., measuring in the blood sample obtained the level of at least one lipid selected from the group of glycerolipids, phospholipids, lysophospholipids, sphingolipids, and cholesterol derivatives, comparing said measured lipid level to that in a predefined standard control, and determining the outcome of a future or existing pregnancy based on said comparison) and if the subject is likely to have a positive pregnancy outcome, performing or continuing with the subject's IVF procedure.

It should be noted that the determination of the lipid levels in the fluid samples according to the methods of the invention may be carried out in any manner known in the art for determining various lipid levels in biological fluids.

According to a specific embodiment, the determination of the lipid levels in a fluid sample may be chemical or enzymatic, for example an assay that hydrolyze/metabolize the lipids, yielding a color or fluorescent label that can be measured, and if desired quantified.

It should be noted that in the effort of determining alterations in the FF lipid network of positive outcome patients, the lipidomics analysis revealed a surprising general trend where high levels of major plasma lipids such as TAGS, DAGs and cholesteryl esters are associated with negative pregnancy outcome, while the accumulation of major membrane lipids such as PLs and SLs is associated with positive outcome.

It is further worth noting that since the total lipid content in the FF is increased in obese women, leading to lipotoxicity, impaired oocyte maturation and early embryonic loss, the methods disclosed herein ought to be used for the assessment of female infertility associated with body weight. Specifically, given that TAG levels inversely correlate to follicle size and positively correlate to maternal BMI and to the levels of adipokines and pro-inflammatory cytokines in FF, the lipid profiling method disclosed in the present invention may be utilized by the skilled practitioner to assess, on the basis of these characteristics and others, the fertility of overweight female subjects.

The skilled practitioner will also appreciate that the novel methods disclosed by the present invention may aid in the initial preliminary diagnosis of a subject's cause of infertility, which could help expedite the course of fertility treatments and reduce the burden felt by the subject and or their partner. As an example of the various applications and permutations of the instant invention that could be contemplated by the skilled practitioner, the assessment in accordance with the method of the instant invention may assign a patient who is seeking fertility treatments to an age-specific treatment group on the basis of, for example, their FF levels of vitamin D derivatives, as significant differences were only demonstrated in younger patients, and hence help to offer a suitable course of treatment for such a patient.

The lipid remodeling demonstrated in the results disclosed herein provides a first detailed characterization of lipid composition in the FF, and thus sheds new light on the association between lipid content and oocyte development. Further to the contribution to basic understanding of the metabolic microenvironment of the oocyte, a skilled practitioner will perceive that these results have the ability to predict pregnancy in various situations with numerous complications that present themselves other than those explicitly demonstrated in the instant invention. Other such predictions may be straightforward and immediate, by using potential markers of positive and negative outcome such as the ones disclosed by the instant invention, and by establishing the correlation between their ratios and pregnancy outcome.

The invention will now be described with reference to specific examples and materials. The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Materials and Methods

### Study Population:

Patients undergoing IVF were recruited at the Assisted Reproductive Technology (ART) center of the Hebrew-University Hadassah Medical Center. The institutional Review Board of Hadassah Medical Organization approved the study (decision number 0207-15-HMO) and each patient signed a consent form before oocyte retrieval. Exclusion criteria included male infertility, or no embryo transfer. Patients underwent controlled ovarian hyper-stimulation by short GnRH agonist protocol or GnRH antagonist protocol as previously described in the art. The ovarian response was assessed by ultra-sound and estradiol (E₂) levels every 2-3 days. Human chorionic gonadotropin or Gonadotropin-releasing hormone agonist or both were administered to induce final oocyte maturation 36 hours before oocyte retrieval. Oocyte retrieval was performed under general anesthesia, using trans-vaginal aspiration with 16-17 gauge needles under ultrasonography guidance. After the oocytes were extracted by an embryologist, the residual FF was pooled and transferred to a laboratory for sample preparation. Fertilization was accomplished by IVF or Intracytoplasmic sperm injection (ICSI). The embryos were cultured in individual wells on a plate in a time-lapse incubator (EmbryoScope™). Embryo transfer was done after 3-6 days according to the embryo morphological grading. Pregnancy test was done two weeks after embryo transfer.

### Sample Preparation for LC-MS Analysis:

Following sample collection, FF samples were immediately centrifuged at 770 g for 10 minutes at 4° C. in order to spin down cells, and the supernatant was collected. Samples were flash-frozen in liquid nitrogen and transferred to −80° C. until analysis. A modified Bligh and Dyer biphasic extraction procedure was used for lipid extraction by slightly acidifying the aqueous phase with the addition of 2% formic acid (GC purity grade) to improve PL yields and protein precipitation (as demonstrated herein further below). Extraction was performed on ice, using ice-cold solvents. 300 μL of FF were thawed and transferred to clean glass tubes. 375 μL of chloroform (UHPLC purity grade) and 750 μL of methanol (LCMS purity grade) were added. Following 30 seconds of vortex, 375 μL of high purity UPLC-MS grade water with 2% formic acid was added. The mixture was vortexed for 30 seconds and then ultra-sonicated for 30 seconds at 4° C., and repeated 5 times for thorough extraction of lipids. Phase separation was carried out by centrifugation at 770 g for 10 min at 4° C. The lower phase containing lipids was transferred to clean glass tubes. Solvents were evaporated in a SC210A SpeedVac concentrator (Thermo Scientific) at 30° C., and dry samples kept at −80° C. until analysis. For liquid chromatography mass spectrometry (LC-MS) run, samples were resuspended in 200 μL 95% acetonitrile (LCMS purity grade)/0.1% formic acid, and then filtered through a 0.22 μm PTFE membrane for subsequent LC-MS analysis.

Ultra-High Performance Liquid Chromatography-Quadrupole Time-of-Flight Mass Spectrometry (UPLC-QTOF-MS):

Lipid analysis was performed using a Waters Acquity UPLC H-Class equipped with a Photodiode Array (PDA) detector, and Xevo X2-XS Q-ToF-High resolution, High Mass Accuracy Q-ToF, equipped with an electrospray ionization (ESI) source. The ESI source was operated in positive (ES+) and negative (ES−) modes in separate acquisitions. A UPLC CSH C18 column (100 mm×2.1 mm, 1.7 μm, Waters, Ireland) was used for the separation of metabolites. The mobile phase consisted of 0.1% formic acid (vol/vol) in water (phase A), 0.1% formic acid (vol/vol) in acetonitrile (phase B), and isopropanol (phase C, LCMS purity grade). The linear gradient program was as follows: From 0 to 1 minute, isocratic flow of 60% A and 40% B. From 1 to 5 minutes, gradient flow where B proportion is increased to 70% (v/v). From 5 to 8 minutes, isocratic flow of 24% A, 40% B and 36% C, from 8 to 9 minutes, isocratic flow of 20% A, 35% B and 45% C, from 9 to 12 minutes, isocratic flow of 18.4% A, 33% B and 48.6% C, from 12 to 17 minutes, isocratic flow of 12% A, 25% B and 63% C, and from 17 to 25 minutes 0.4% A, 10.5% B and 89.1% C. From 25.51 to 35 minutes, the system was allowed to re-equilibrate to the initial conditions. Following preliminary experiments, the retention time of 1.0-25 min was used for analysis. The flow rate was 0.4 mL/min, and the column temperature was kept at 60° C. Capillary spray was maintained at 3.0 kV, cone voltage at 40 eV, and collision energy at 15 eV. All data was acquired through MS^{E} analyses, collision energy was 40-65 eV for positive mode and 30-60 eV for negative mode. Full-scan and MS^{E} mass spectra were acquired from 30-2,000 Daltons. Argon was used as the collision gas for collision induced dissociation. The mass spectrometer was calibrated using sodium formate, and leucine enkephalin was used as the lock mass (m/z 556.2771, 200 pg mL-1) and continuously infused at 6 μL/min, and data was acquired from (ES+) mode. MassLynx software version 4.1 (Waters) was used to control the instrument and calculate accurate masses. Post column derivatization was employed with ammonium fluoride to improve the yields of neutral charged lipids in the (ES+) mode as [M+NH₄]⁺. A solution of 1 mM ammonium fluoride in 50:50 methanol:water was automatically continuously injected into the MS together for post column derivatization in Positive mode runs, to improve the yields of neutral charged lipids.

Two quality control sets were used to assure data quality: 1) A pool of all samples from the current analysis was injected after every 10 samples. 2) A second sample containing 9 lipid standards [arachidonic acid 5 μM; N-hexanoyl-D-sphingosine10 μM; stearoyl-SN-glycero-3-phosphocholine 10 μM; 24:0 C24 ceramide-1-phosphate (d18:1/24:0) 10 μM; Triglyceride Mix (a mix of 5 standards—Triacetin (C2:0)5, Tributyrin (C4:0), Tricaproin (C6:0), Tricaprylin (C8:0), Tricaprin (C10:0); Sigma-Aldrich)] was injected every other 10 samples.

A standard was used to validate the identification of 25-hydroxyvitamin D3 monohydrate (Sigma-Aldrich).

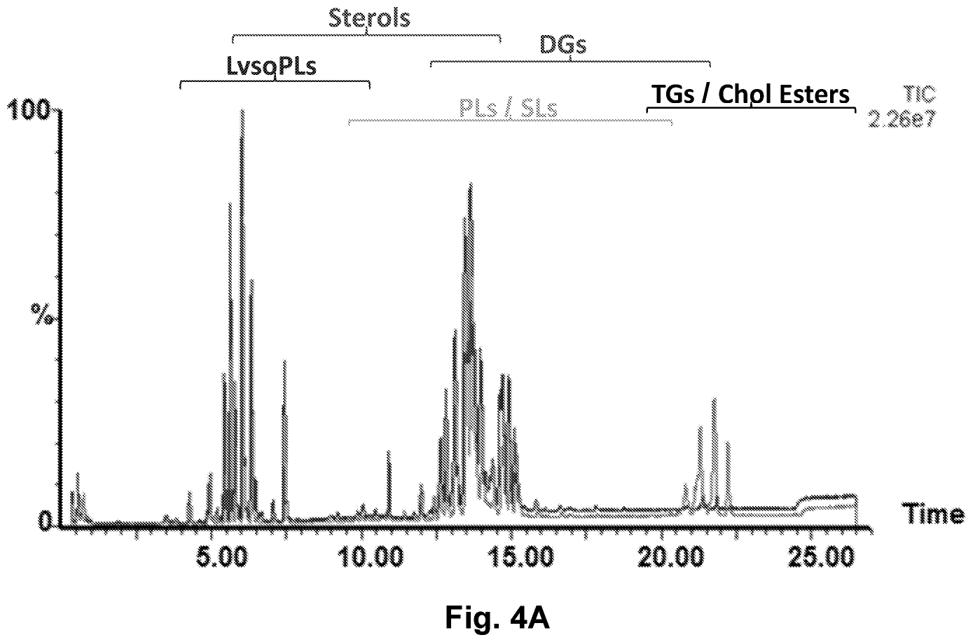

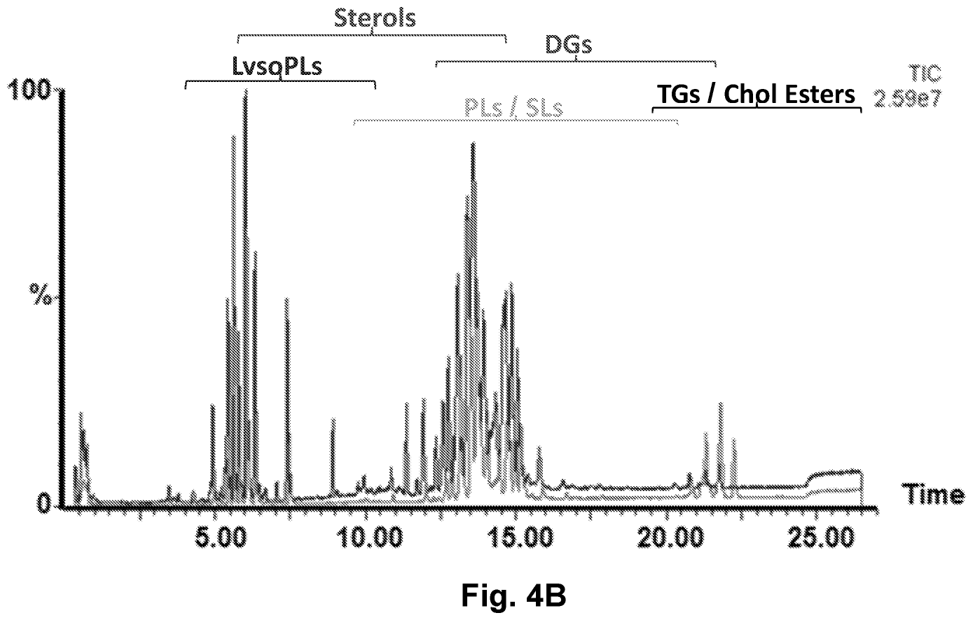

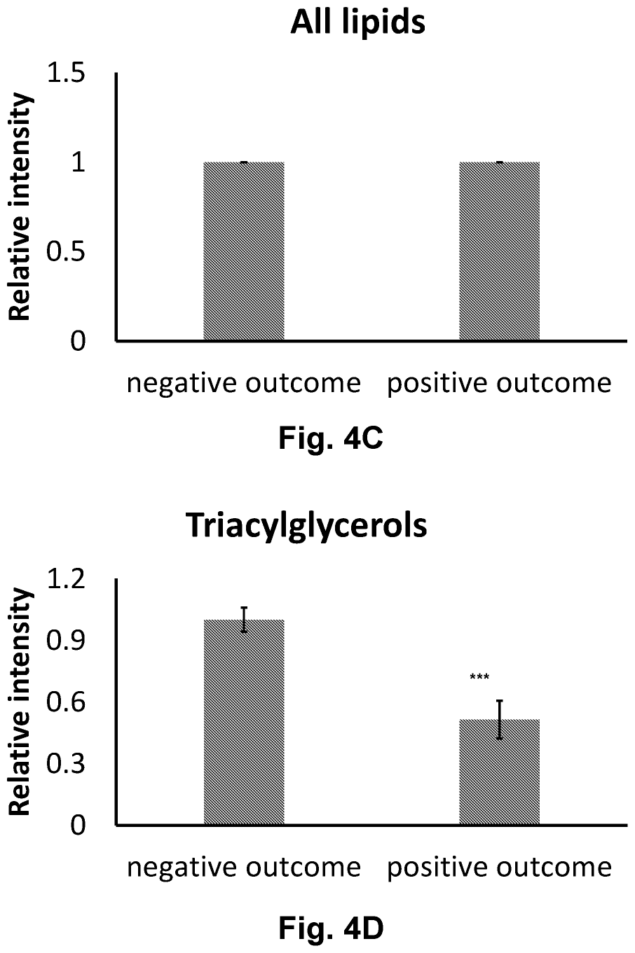

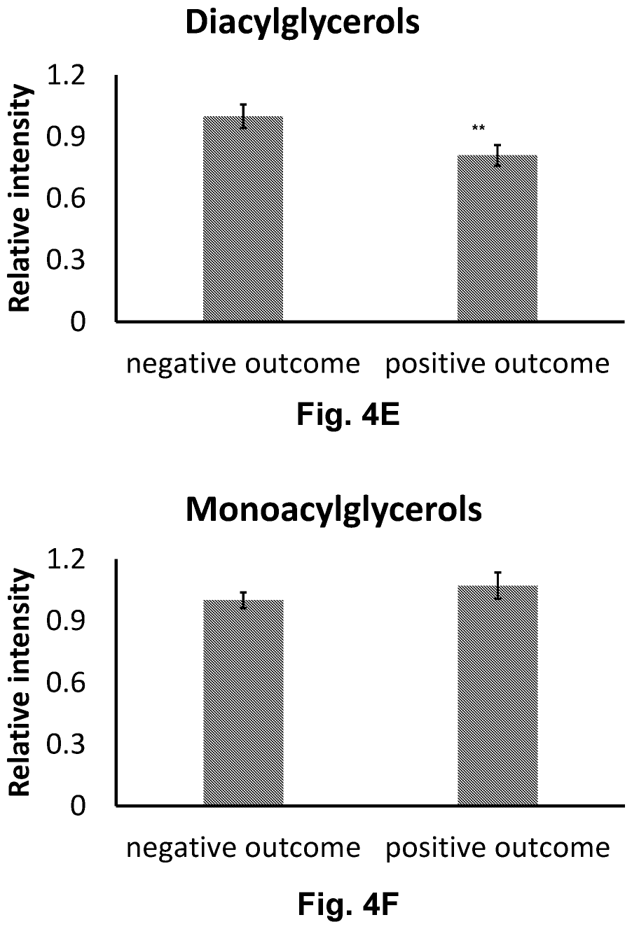

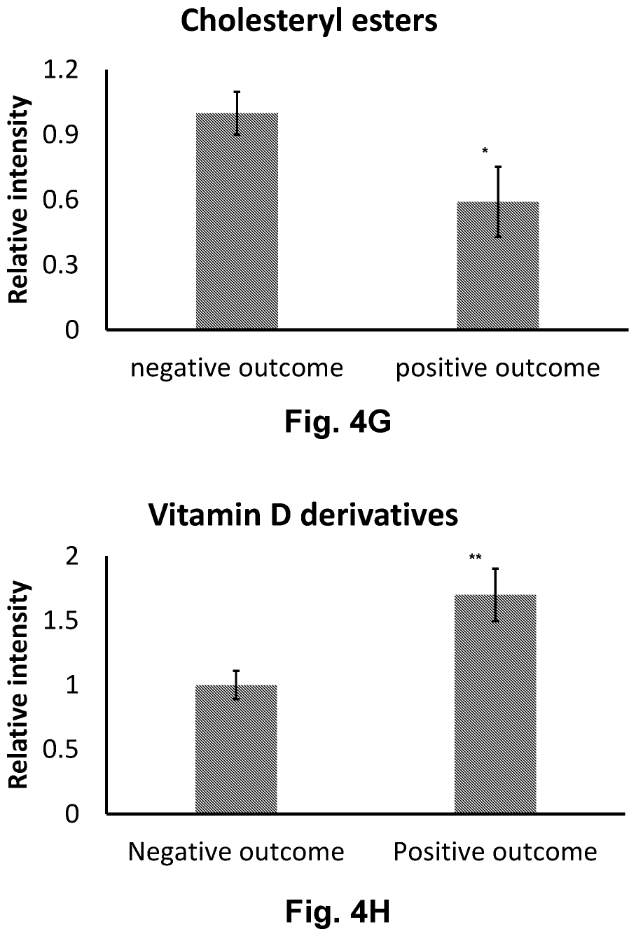

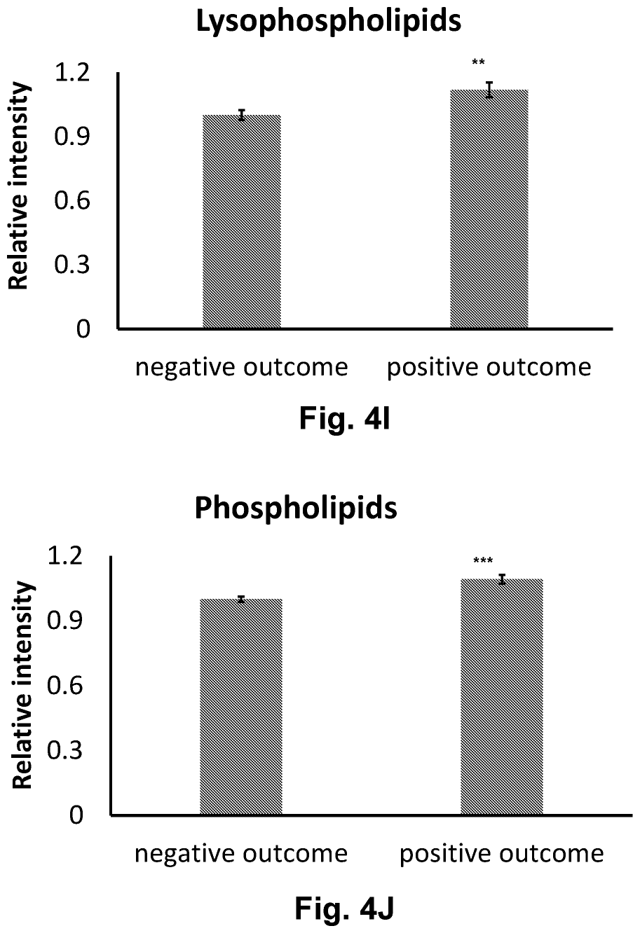

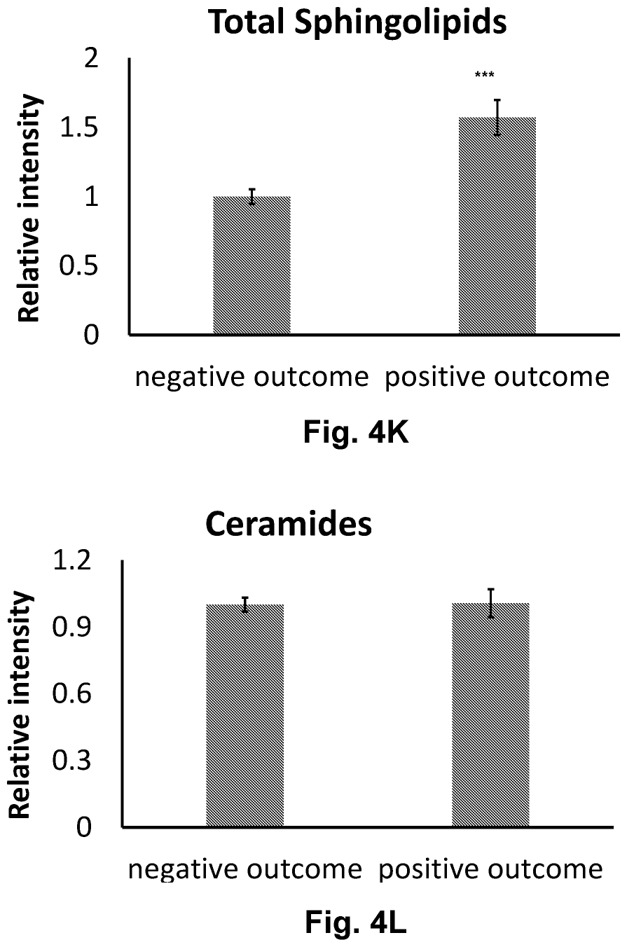

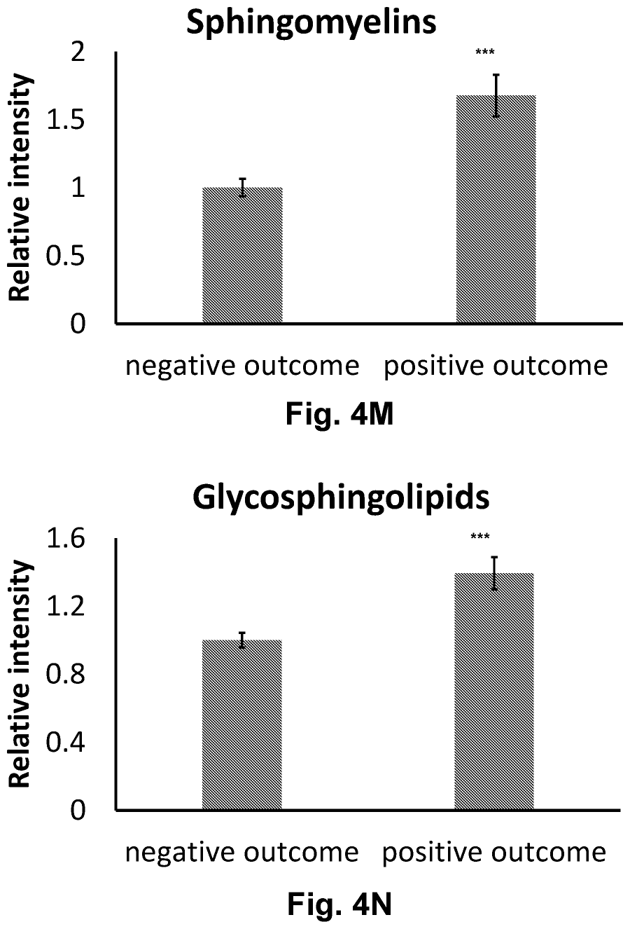

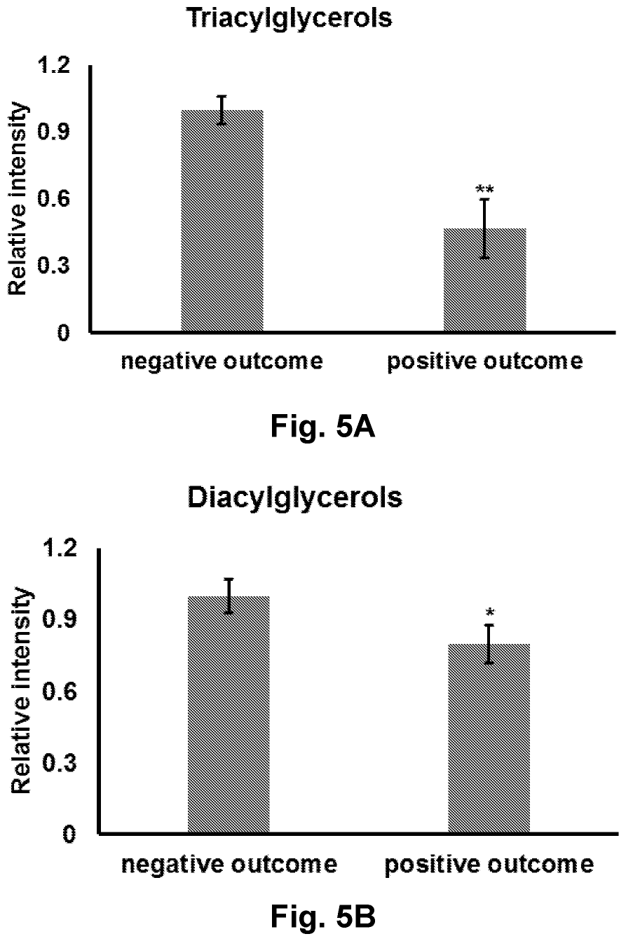

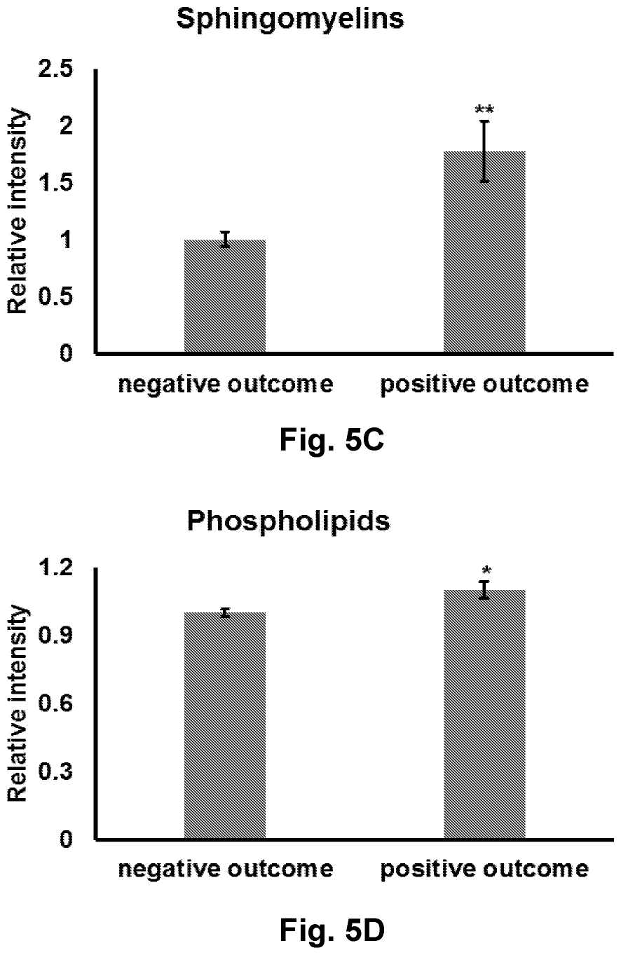

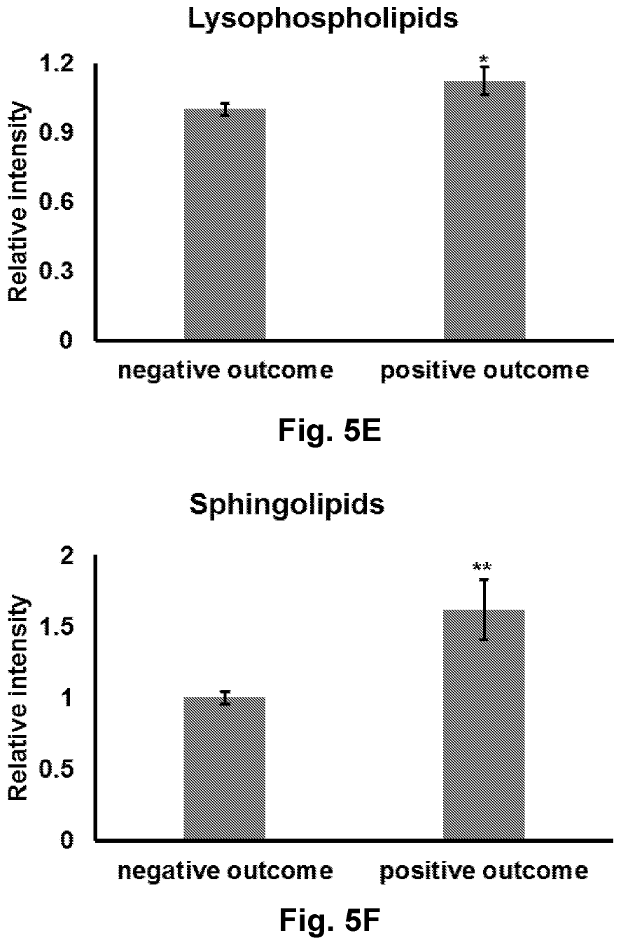

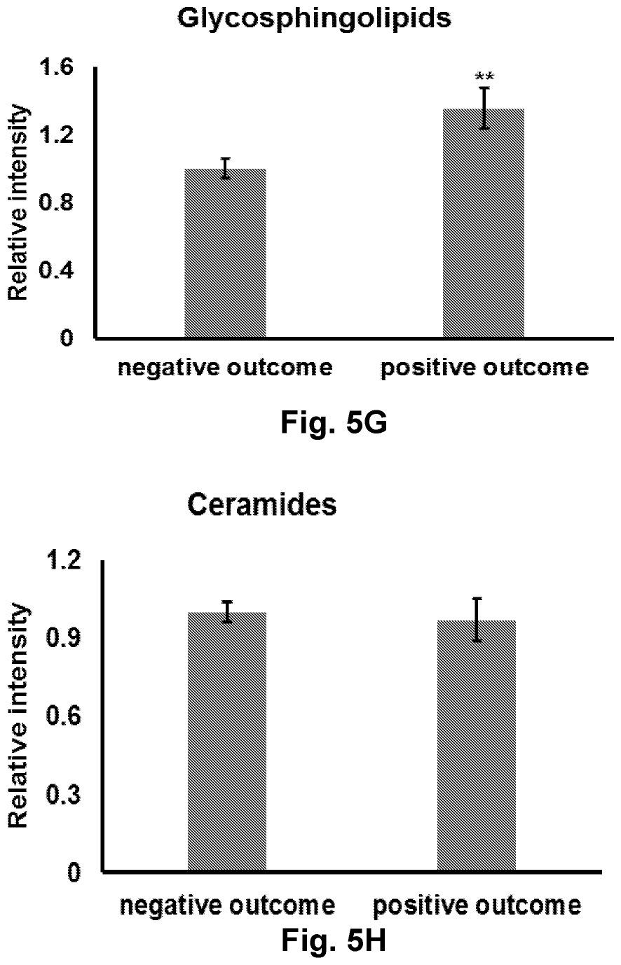

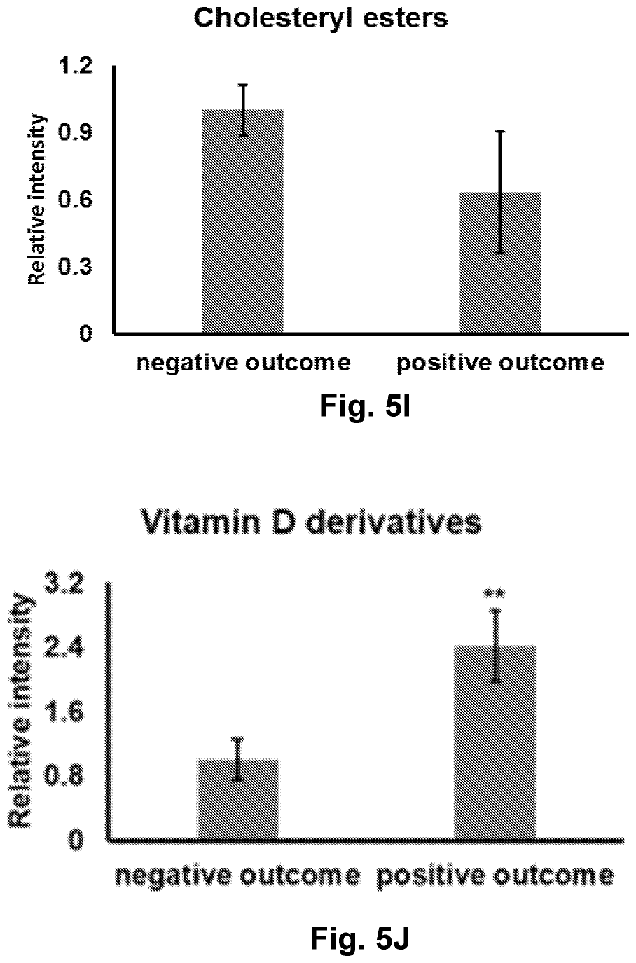

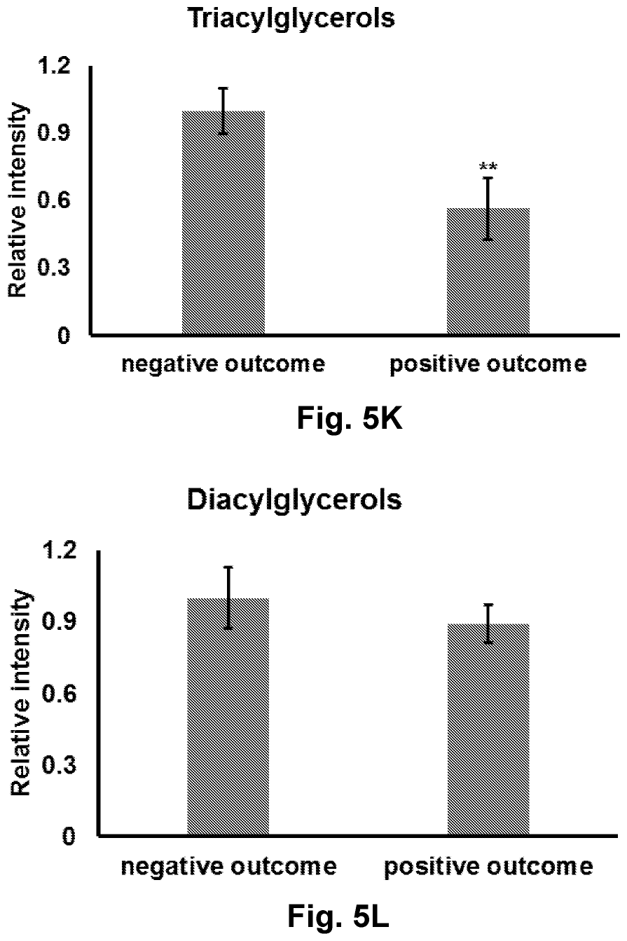

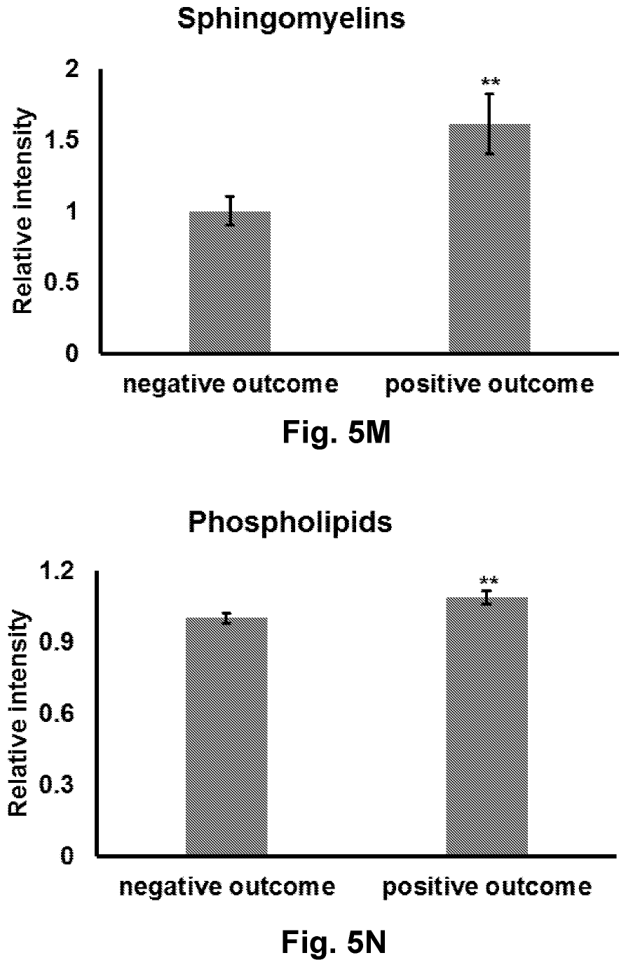

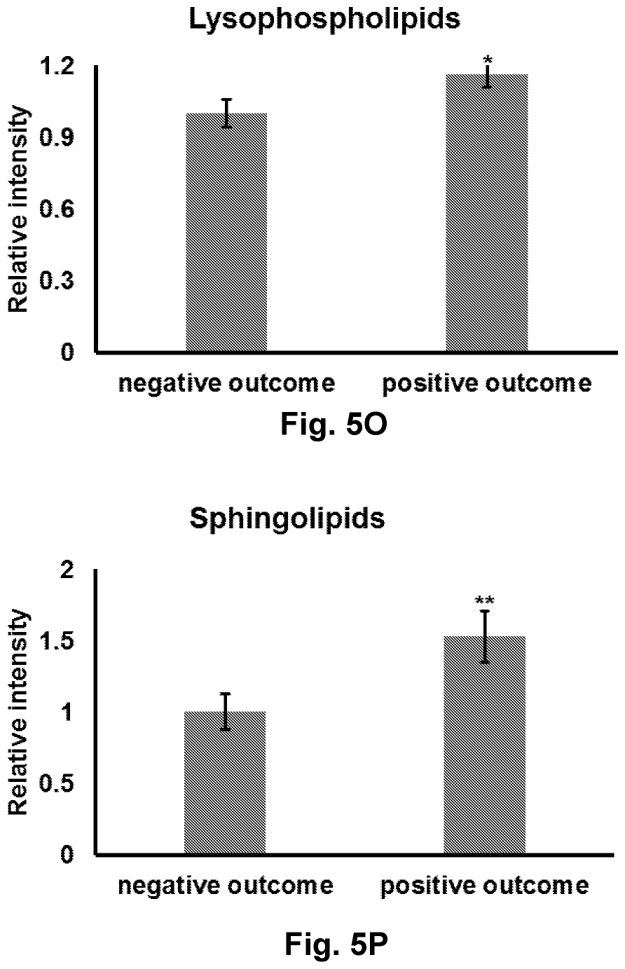

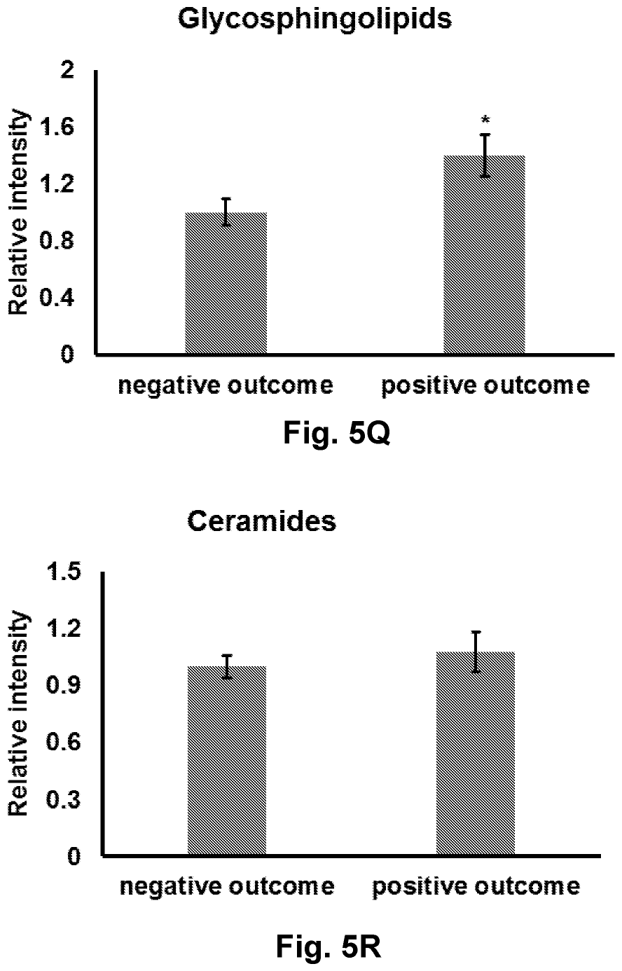

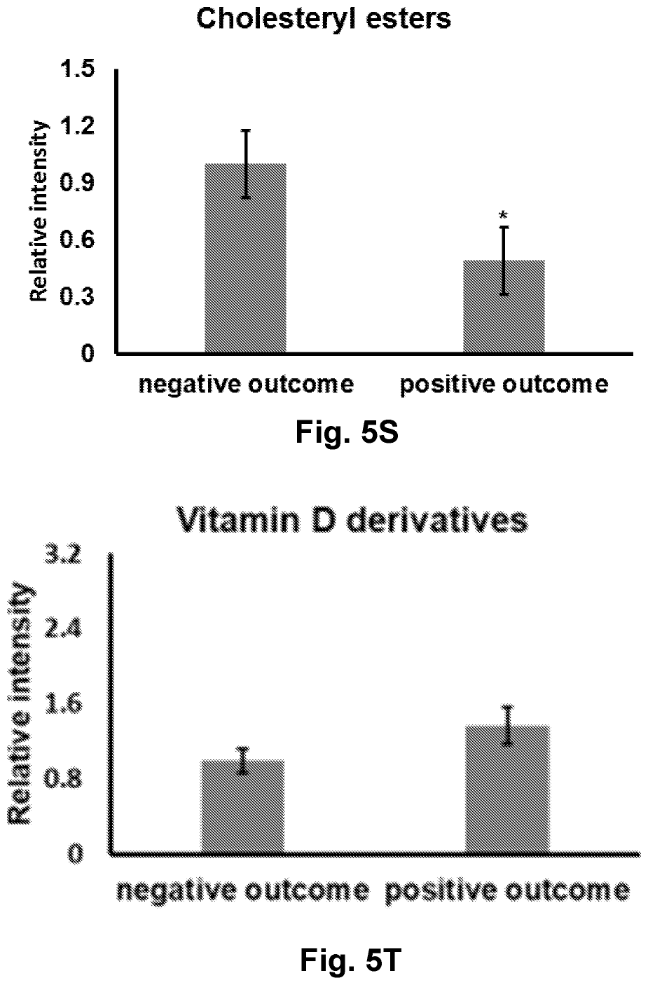

MassLynx 4.1 (Waters Co., UK) was used for mass spectra visualization and Progenesis QI (Nonlinear dynamics, UK) for spectra deconvolution, alignment, normalization and identification. To exclude masses that did not originate from FF samples, blank samples (solvents that went through sample preparation, but contained no FF) were injected. Masses with a minimum mass-to-charge ratio cutoff of 100 m/z, lowest mean abundance in blank, and fold change (FC) over 100 from blank were used for analysis. The generation of partial least squares discriminant analysis (PLS-DA) and a heat map and the corresponding analyses (permutation test and volcano plot) were carried out using MetaboAnalyst 4.0. For multivariate tests, Range Scaling was used in order to eliminate the dependence of the rank of the lipids on their measured abundance. MS^{E} was used for exact mass acquisition of precursor and fragment ion spectra from every detectable component of the samples. Lipid identification was then carried out according to exact mass (mass accuracy<5 PPMs, retention time (different lipid groups have typical retention times—see FIGS. 4A-4B), isotope pattern and fragmentation pattern. Data from all lipids were compared against 18 metabolite libraries compatible with Progenesis QI for the putative annotation of lipids. The exact mass, isotopic pattern and fragmentation pattern of lipids were then further validated against theoretical data). MS/MS experiments were carried out to further validate the identities of 32 differential lipids [fold change>3 and false discovery rate (FDR) adjusted p value≤0.05 from the (ES+) data set]. A table of the exact masses, retention time, MS fragments and putative identification of these differential lipids is provided as Table 2. A Receiver operating characteristic (ROC) curve was generated according to the data of the 6 potential biomarkers suggested in FIG. 5B, using Matlab (Version R2017a). Prediction accuracy, prediction sensitivity and specificity were calculated.

### Example 1

Optimization of the Lipid Extraction System from Follicular Fluid

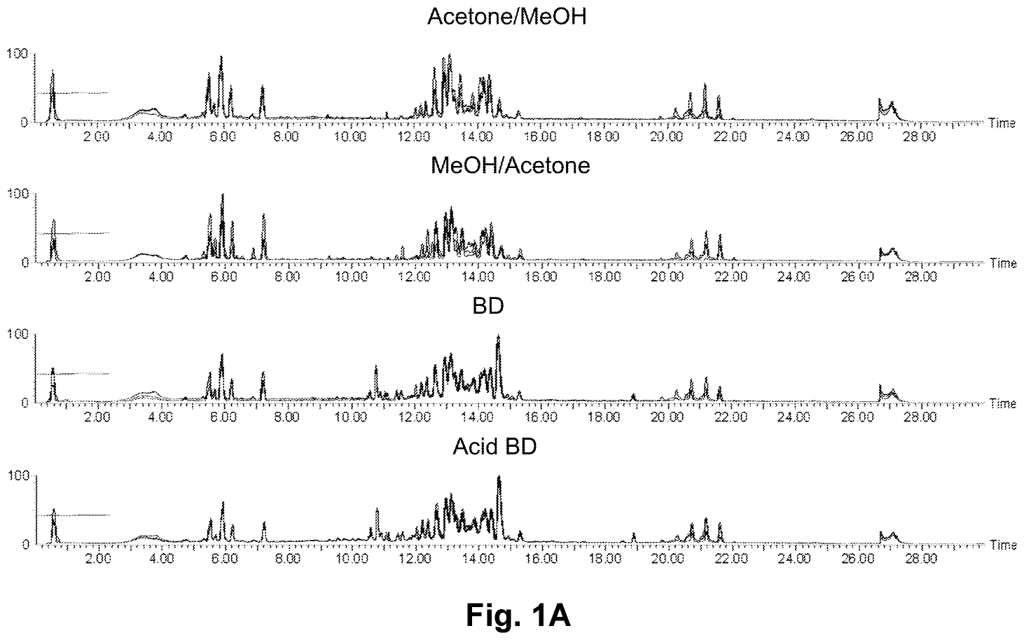

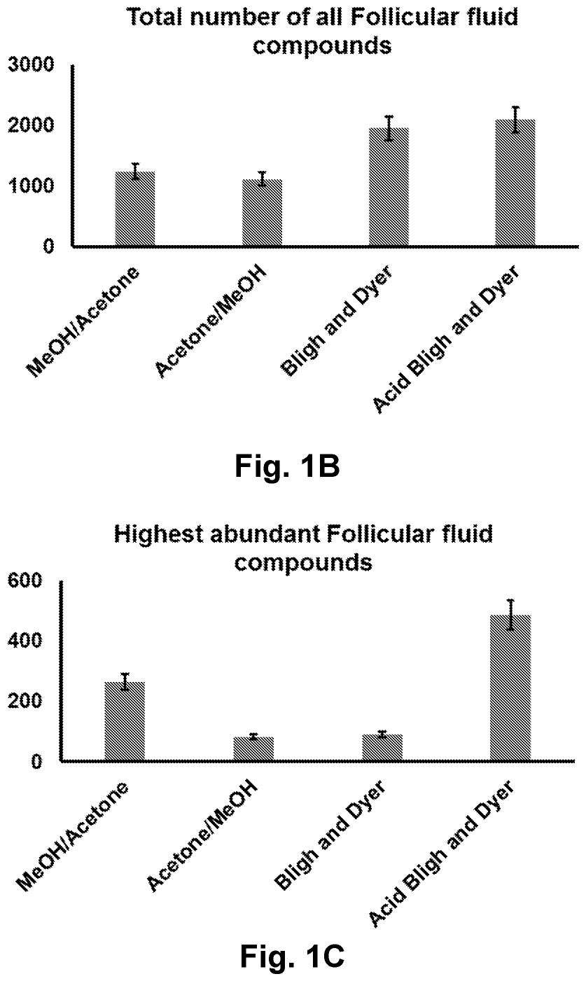

A specific objective of the instant invention is to define the metabolic alterations in the microenvironment of the oocyte that are associated with pregnancy outcome, and that may influence oocyte development. To optimize sample preparation for the highest yield of metabolites from the FF, different extraction systems were tested. Surprisingly, chloroform extraction of the aqueous FF yielded the highest total number of metabolites, and the highest number of most abundant metabolites (FIGS. 1A-1C), suggesting that lipids constitute a major component of the FF metabolome. The lipid extraction procedure from the FF was further optimized by a mild acidification that resulted in increased yields of lipids (FIGS. 1B and 1C). This extraction system was therefore utilized for the preparation of samples of the main cohort of IVF patients.

### Example 2

Clinical Data and Preliminary Assessment of FF Lipid Profiles from Women Undergoing IVF

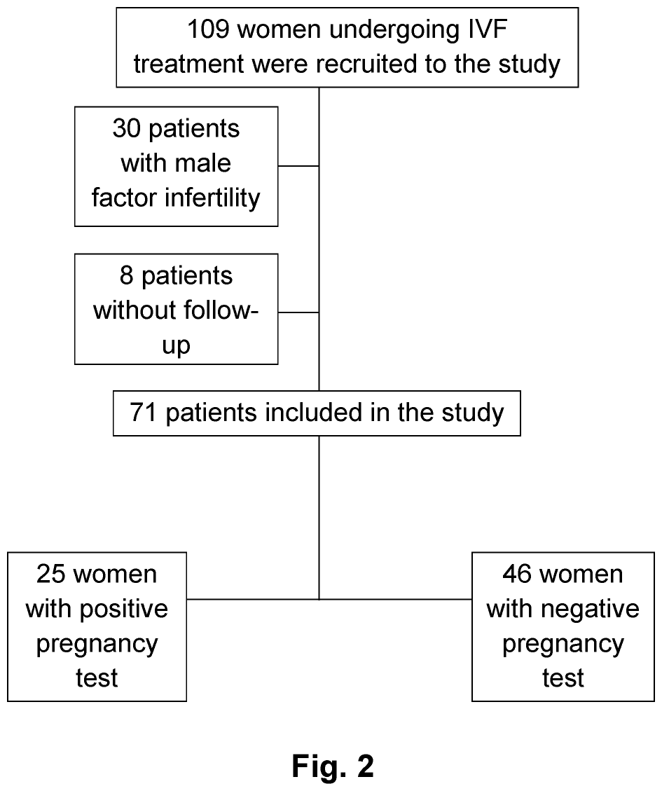

A total of 109 women underwent fresh embryo transfer with US guidance. After the exclusion of patients with male factor background, or unknown pregnancy outcome, the FF lipid composition of 71 patients (FIG. 2) was taken for lipidomics analysis. Demographic and gynecologic features as well as IVF treatment—related data are presented in Table 3. As expected, the clinical data points to differences between the positive and the negative outcome groups in the age and BMI of the patients.

TABLE 3
Patient characteristics, IVF protocol, and pregnancy outcome

| | Pregnancy + | Pregnancy − | P |
|---|---|---|---|
| Characteristic | (n = 25) | (n = 46) | value |
| Age (yr) | 34.8 ± 7.1 | 38.2 ± 5.1 | 0.02 |
| BMI (kg\m²) | 23.1 ± 6.6 | 27.4 ± 6.8 | 0.01 |
| Gestation | 1.2 ± 1.2 | 0.8 ± 1.2 | 0.26 |
| Deliveries | 0.6 ± 0.9 | 0.2 ± 0.5 | 0.05 |
| Miscarriages | 0.5 ± 0.8 | 0.5 ± 0.9 | 0.93 |
| Infertility diagnosis | | | |
| Ovulation | 4 | 7 | 0.93 |
| dysfunction | | | |
| Mechanical factor | 4 | 5 | 0.53 |
| Unexplained | 12 | 25 | 0.61 |
| infertility | | | |
| PGD | 3 | 4 | 0.66 |
| No. of cycles | 1.8 ± 1.4 | 2.0 ± 1.3 | 0.54 |
| Protocol | | | |
| Antagonist | 17 | 27 | 0.44 |
| Short agonist | 8 | 17 | 0.68 |
| Natural | | 1 | — |
| Long protocol | | 1 | — |
| No. of follicles | 11.2 ± 5.7 | 8.5 ± 6.4 | 0.09 |
| E2 max (pM) | 6356 ± 2633 | 5795 ± 3188 | 0.46 |
| Oocytes no. | 10.2 ± 6.0 | 8.7 ± 8.0 | 0.40 |

The lipidomics study resulted in 9953 features detected (ES+). Following stringent exclusion of possible artifactual features arising from masses that match those found in blank samples (see Materials and Methods section), 1571 features were assigned as FF lipids. 1032 of these were putatively identified. 11403 features were detected using (ES−), of which, 1787 were assigned as FF lipids, and 828 metabolites putatively identified. The (ES+) data set was chosen for further analyses based on the high number and versatile nature of the lipids that were putatively identified using Progenesis QI, as these suggested a potentially global and deep lipid profile. The identification of 32 lipids that showed the most discriminative accumulation (>3 fold change in abundance between positive and negative outcome samples, and p value≤0.05 after the adjustment for false discovery rate (FDR)) was further validated by MS/MS experiments.

### Example 3

Statistical Analyses Show that Lipid Composition from Positive Outcome FF is Distinct from that of Negative Outcome FF

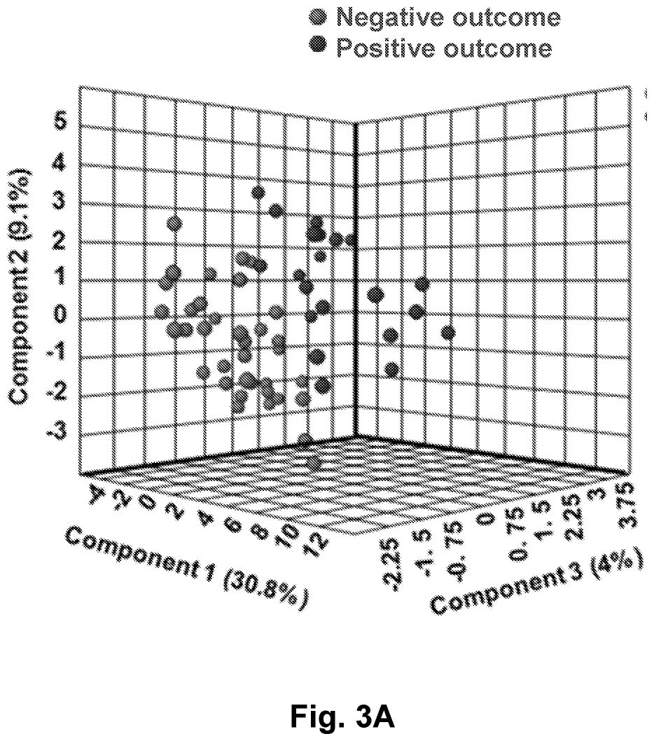

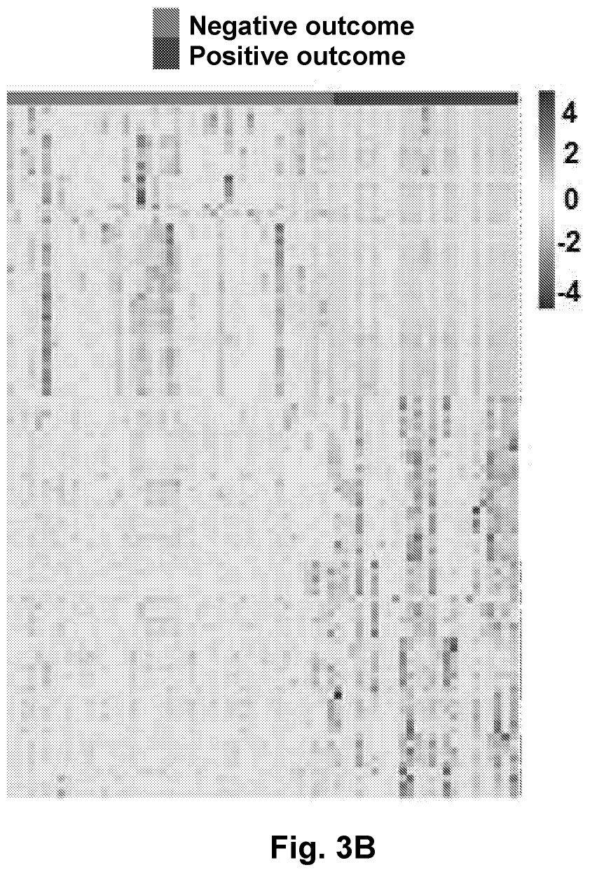

A partial least squares discriminant analysis (PLS-DA) of all FF-originated features (1571) showed a separation between the FF lipid composition of positive and negative outcome patients (FIG. 3A; R²=0.83 and Q²=0.47). To address a possible overfit, a permutation test was performed with 1000 permutations, suggesting prediction accuracy during training of empirical p value: p<0.001. The alterations in the lipid composition of positive outcome patients is underscored by a heat map of the top 100 discriminative lipids (based on t-test) between positive and negative outcome FF samples (FIG. 3B).

### Example 4

### Lipid Profiling Demonstrates a Lipid Remodeling Throughout Different Biosynthetic Lipid Groups in the FF of Positive Outcome Patients

To better understand the nature of the lipid signature and metabolic processes responsible for the separation of the FF lipidome of positive and negative outcome patients, the lipids in the list of variable importance in projection (VIP) were grouped. This list demonstrated a lipid remodeling throughout different biosynthetic lipid groups, suggesting shifts in the metabolism of TAGS, DAGs, PLs, Lysophospholipids, SLs, sphingomyelins, cholesteryl esters and vitamin D derivatives. Within the characteristic retention time-frames, differences between positive and negative outcome FF could be seen even in the total ion current (TIC) chromatograms (FIGS. 4A and 4B). The abundances of lipids from each of these biosynthetic groups in the FF of patients with positive and negative outcomes were quantified relative to one another. The accumulations of total lipid species detected in all samples (after the exclusion of possible artifactual features) were compared and no difference was found between samples from positive and negative outcome patients (FIG. 4C). However, changes across different lipid biosynthetic groups were observed. Importantly, while the accumulation of several lipid groups was lower in the positive outcome FF, the accumulation of others was higher (FIG. 4D-4N). The most striking difference in the lipid composition of positive and negative outcome FF was the lower accumulation of TAGs in positive outcome patients (FIG. 4D). The levels of diacylglycerols (DAGs) in the FF also showed an inverse correlation to pregnancy outcome, with milder but still highly significant differences (FIG. 4E), whereas no difference was noted in the accumulation of monoacylglygerols (MAGs; FIG. 4F). In addition, changes in the accumulation of cholesterol derivatives (cholesteryl esters) and vitamin D derivatives were observed. Interestingly, while the accumulation of cholesteryl esters were lower in the positive outcome FF (FIG. 4G), the levels of vitamin D derivatives were higher (FIG. 4H), suggesting metabolic shift or shifts that take place downstream of cholesterol synthesis.

Given the conflicting reports regarding association between the accumulation of vitamin D in the FF and pregnancy rates, the inventors further validated the identification of 25-hydroxy vitamin D, the major vitamin D metabolite, by use of an authentic standard. While the higher accumulation of 25-hydroxy vitamin D in positive outcome FF did not reach significance, the total accumulation of all vitamin D derivatives was significantly higher in the positive outcome FF.

Lysophospholipids and PLs (FIG. 4I-J) showed higher accumulation in positive outcome patients. The change in the levels of PLs is especially notable, given the tight regulation of their biosynthesis and abundance, which is necessary for maintaining homeostasis.

The total abundance of another group of membrane lipids—SL species, was also higher in positive outcome FF (FIG. 4K). As SLs constitute an extremely versatile group of lipids with great structural and functional diversity, the inventors studied the accumulation of notable sub-groups of SLs. No difference was noted in the levels of ceramides—the simplest SL species (composed of sphingosine and a fatty acid; FIG. 4L). In contrast, sphingomyelins (also classified as sphingophospholipids; FIG. 4M), and glycosphingolipids (sphingolipids with attached carbohydrate chains; FIG. 4N) followed the accumulation pattern of total SLs, with higher abundance in the positive outcome FF.

To exclude a possible age-related effect on lipid composition, which may be independent of pregnancy outcome, the accumulation of the same lipid groups in the FF of positive and negative outcome patients was compared within each respective age group. The results obtained are similar to those obtained with the dataset that includes all age groups, with the exception of vitamin D accumulation in older patients, which showed no difference in FF from positive and negative outcome patients (FIGS. 5A-5T).

### Example 5

### Determination of Lipid Biomarkers for Predicting Successful Pregnancy Outcome

Finally, to pinpoint specific lipids with the most distinguishable accumulation characteristics in positive outcome FF, both the fold change (FC) and the statistical evaluation (in the form of FDR adjusted p values) of the abundance of specific lipid species in positive and negative outcome FF were determined. These are presented as a volcano plot, that suggested 32 most discriminant lipids [FC>3 and a FDR adjusted p value≤0.05; FIG. 6A). The list of lipids that demonstrated the highest FC together with significant p values consisted of TAGS, PLs, lysophospholipids and one sphingomyelin (FIG. 6A).

Further analyses were pursued to provide a possible, straightforward and immediate MS-based assessment of the pregnancy potential of a given patient. By examining the accumulation of several selected lipids, which may be used as biomarkers, the distinction of positive outcome FF became apparent by their extracted ion chromatograms, even with no bio-statistical processing (FIGS. 6B-6G).

A receiver operating characteristic (ROC) curve analysis of the predictive ability of 6 of the potential lipid biomarkers resulted in a high diagnostic performance, with 86% area under curve (AUC) (FIG. 6H).

Altogether, the data presented provide a FF lipid signature of the outcome of pregnancy that may be easily and immediately determined.

1. A method for determining the likelihood of a positive pregnancy outcome in a subject before or during fertilization treatment, the method comprising:
a) obtaining a fluid sample from a biological entity being the subject or an oocyte thereof;
b) measuring in the fluid sample obtained in step (a) the level of at least one lipid selected from the group of glycerolipids, phospholipids, lysophospholipids, sphingolipids, and cholesterol derivatives;
c) comparing the level of the at least one lipid measured in step (b) to the level of said at least one lipid in a predefined standard control; and
d) determining the outcome of future or existing pregnancy based on the comparison in (c).

2. The method according to claim 1, further comprising the following step:
e) diagnosing the source of infertility for further treatment.

3. The method according to claim 1, further comprising the following step:
f) the subject with a suitable treatment.

4. The method according to claim 1, wherein step (b) further comprises measuring the levels of at least one of vitamin D, a derivative of vitamin D, several derivatives of vitamin D, or a combination of vitamin D and one or more of its derivatives.

5. The method according to claim 1, wherein step (b) further comprises measuring lipid levels from at least two different lipid groups, optionally at least three, optionally at least four, optionally of all five groups.

6. The method according to claim 1, wherein step (c) further comprises integrating the lipid levels from at least two, optionally at least three, optionally at least four, optionally from all five of the lipid groups, relative to predefined standard control levels, to provide a score reflecting the overall change across the lipid profile.

7. The method according to claim 1, wherein the subject is a human or a mammal.

8. The method according to claim 7, wherein the human subject has no history of infertility, unexplained infertility, known female infertility, known male factor infertility or is undergoing fertility treatment.

9. The method according to claim 1, wherein the biological entity is a fertilized oocyte originating from said subject or another subject, or is genetically engineered oocyte.

10. The method according to claim 1, wherein a pregnancy outcome is considered positive if the female subject becomes pregnant, an oocyte develops into a viable fetus, the fetus continues to develop until its successful delivery, the pregnancy holds to full term, or the cycle of in vitro fertilization (IVF) is successful.

11. (canceled)

12. The method according to claim 1, wherein the fluid sample obtained from the biological entity is selected from: follicular fluid (FF) of a single oocyte; a pool of follicular fluids of several oocytes; and blood or plasma of a female subject that is either pregnant or is seeking to be pregnant in the future.

13. The method according to claim 3, wherein said suitable treatment is selected from: selecting a fertilized oocyte for implantation, implanting a fertilized oocyte in a subject, starting or continuing with an IVF treatment cycle, treating said subject or their male partner for infertility, and using a surrogate mother for the pregnancy.

14. The method according to claim 1, wherein a positive pregnancy outcome is indicated when the measured levels of at least one lipid selected from glycerolipids and cholesterol derivatives in said fluid sample are low relative to those of a predefined negative pregnancy outcome standard control.

15. The method according to claim 14, wherein a positive pregnancy outcome is indicated when the fold change in the level of at least one lipid selected from glycerolipids and cholesteryl ester, measured in said fluid sample is 10% less relative to that of a predefined negative pregnancy outcome standard control.

16. The method according to claim 14, wherein the glycerolipids are triacylglycerol (TAG), diacylglycerol (DAG), or a combination thereof.

17. The method according to claim 1, wherein a positive pregnancy outcome is indicated when the measured levels of at least one lipid selected from phospholipids, lysophospholipids, sphingolipids, and vitamin D derivatives, are high in said fluid sample relative to that of a predefined negative pregnancy outcome standard control.

18. The method according to claim 17, wherein a positive pregnancy outcome is indicated when the fold change in the level of at least one lipid selected from phospholipids, lysophospholipids, sphingolipids, or vitamin D derivatives, measured in said fluid sample is 10% more relative to that of a predefined negative pregnancy outcome standard control.

19. The method according to claim 1, wherein a positive pregnancy outcome is indicated when:
the fold change in a level of a lipid selected from: LysoPC(18:0), LysoPC(18:1), SM(d18:1/16:0), Lyso PC(18:1), LysoPC(18:2), PC(P-16:0/20:2) is at least 10% less than that of a predefined negative pregnancy outcome standard control, or
the fold change in a level of a lipid selected from: TG(15:1/24:1/18:2), TG(14:1/16:0/20:0), TG(18:1/14:0/22:1), TG(14:1/20:0/21:0), TG(14:0/18:3/16:0), TG(18:0/24:0/20:4), TG(14:1/19:0/22:1), TG(18:0/16:0/18:0), TG(16:0/16:1/16:1), TG(20:0/20:3/22:0), TG(20:0/22:3/22:2), TG(16:1/18:0/20:0), TG(16:0/16:0/16:1), TG(14:0/16:0/16:1), TG(18:1/16:0/18:0), TG(16:1/18:1/18:1) is at least 10% greater than that of a predefined negative pregnancy outcome standard control.

20-21. (canceled)

22. A method for selecting an oocyte suitable for use in IVF treatment from a plurality of candidate oocytes, the method comprising:
a) obtaining follicular fluid of each candidate oocyte;
b) measuring in the follicular fluid obtained in step (a) the level of at least one lipid selected from the group of glycerolipids, phospholipids, lysophospholipids, sphingolipids, and cholesterol derivatives;
c) comparing the level of the at least one lipid measured in step (b) to the level of said at least one lipid in a predefined standard control;
d) identifying one or more oocytes likely to result in a positive pregnancy outcome based on the comparison in (c); and
e) selecting one or more of the oocytes identified in step (d) for use in IVF treatment.

23. A method for determining whether to perform or continue an IVF procedure in a subject, the method comprising:
a) obtaining a blood sample from a subject;
b) measuring in the blood sample obtained in step (a) the level of at least one lipid selected from the group of glycerolipids, phospholipids, lysophospholipids, sphingolipids, and cholesterol derivatives;
c) comparing the level of the at least one lipid measured in step (b) to the level of said at least one lipid in a predefined standard control; and
d) determining the outcome of future or existing pregnancy based on the comparison in (c);
wherein if said subject is likely to have a positive pregnancy outcome, performing or continuing with the subject's IVF procedure.
